(19) United States
(12) Patent Application Publication
(10) Pub. No.: US 2022/0315933 A1
(43) Pub. Date: Oct. 6, 2022

(54) SYSTEMS AND METHODS FOR SINGLE-STRAND BREAK SIGNALING AND REPAIR IN A CELL-FREE SYSTEM AND METHODS OF IDENTIFYING MODULATORS OF SINGLE-STRAND BREAK SIGNALING AND REPAIR

(71) Applicant: University of North Carolina Charlotte, Charlotte, NC (US)

(72) Inventor: Shan YAN, Charlotte, NC (US)

(21) Appl. No.: 17/726,156

(22) Filed: Apr. 21, 2022

Related U.S. Application Data
(62) Division of application No. 16/491,037, filed on Sep. 4, 2019.
(60) Provisional application No. 62/467,894, filed on Mar. 7, 2017.

(51) Int. Cl. C12N 15/63; C12N 9/22; C12N 15/10; G01N 33/573; C12Q 1/48; C12N 15/11

(52) U.S. Cl. CPC C12N 15/63; C12N 9/22; C12N 15/1093; G01N 33/573; C12Q 1/485; C12N 15/11; C12N 2800/10; C12N 2800/70; C12N 2999/005; G01N 2440/14; G01N 2500/00; C12N 2310/533; G01N 2500/20; C12N 2310/532

(57) ABSTRACT

The present application describes structures, systems, and methods for modeling and analysis of single-strand break (SSB) signaling and repair in a cell-free system. Also provided are methods of making the SSB structures and SSB signaling and repair systems. Methods and systems for identifying modulators of DNA damage response (DDR) activity for SSB repair are also described as well as methods of inhibiting SSB repair.

### CROSS-REFERENCE TO RELATED APPLICATIONS

This application is a divisional of U.S. application Ser. No. 16/491,037 having the title “Systems and Methods for Single-Strand Break Signaling and Repair in a Cell-Free System” filed Sep. 4, 2019, which is the 35 U.S.C. § 371 national stage application of PCT Application No. PCT/US2017/065639, having the same title, filed Dec. 11, 2017, which claims the benefit of and priority to U.S. Provisional Application Ser. No. 62/467,894, having the title “Single Strand Break Signaling in a Cell-Free System”, filed on Mar. 7, 2017, both of which are incorporated herein by reference in their entireties.

### STATEMENT REGARDING FEDERALLY SPONSORED RESEARCH OR DEVELOPMENT

This invention was made with government support under GM114713 awarded by National Institutes of Health. The U.S. government has certain rights in this invention.

### SEQUENCE LISTING

This application contains a sequence listing filed in electronic form as an ASCII.txt file entitled 222118-2020_ST25.txt, created on Dec. 11, 2017 and having a size of 14 KB. The content of the sequence listing is incorporated herein in its entirety.

### BACKGROUND

DNA single-strand breaks (SSBs) are generated approximately 10,000 time per day per mammalian cell and considered as the most common type of DNA damage. SSBs result from various cellular processes, including, but not limited to, unbalanced reactive oxygen species, intermediate products of DNA repair pathways such as base excision repair (BER), and aborted activity of cellular enzymes such as Topoisomerase 1 (Top1) (Caldecott, 2008; Yan et al., 2014). If not repaired properly or promptly, SSBs lead to genome instability and have been associated with the pathologies of cancer and neurodegenerative disorders (Caldecott, 2008; Yan et al., 2014). However, it remains unknown how cells sense and recognize unrepaired SSBs in their genome to trigger a DNA damage response (DDR) pathway.

Several critical barriers hinder understanding of SSB signaling at molecular level. The first critical barrier for SSB signaling is the lack of a defined experimental system to dissect all aspects of SSB signaling. Cellular signaling to double-strand breaks (DSBs) has been studied via generating a single site-specific DSB in a genome by HO or I-Scel endonuclease in yeast and mammalian cells (Costelloe et al., 2012; Hicks et al., 2011; Richardson and Jasin, 2000; Rudin and Haber, 1988). Current understanding of SSB signaling comes primarily from an experimental system using indirect generation of SSBs after treatment of exogenous reagents such as hydrogen peroxide or methyl methanesulfonate (MMS) (Khoronenkova and Dianov, 2015; Willis et al., 2013). Spatial and temporal cellular response to multiple SSBs induced by UVDE (UV damage endonuclease) was characterized in human cells (Okano et al., 2003). The second critical barrier for SSB signaling is the inability in existing systems to distinguish SSBs from DSBs. Many DNA damaging reagents generate both SSBs and DSBs simultaneously or sequentially. Thus, with current technology, it is extremely difficult to directly explore SSB signaling exclusively in response to SSBs, as opposed to a combination of SSBs and DSBs. Thus, it remains unknown whether a defined SSB structure triggers a specific SSB signaling for a DDR pathway.

### SUMMARY

In various embodiments, the present disclosure provides defined site-specific single-strand break (SSB) plasmid structure that can trigger an SSB DNA damage response (DDR) pathway in a eukaryotic cell-free system, as well as systems and kits including the SSB plasmid structure and methods of making the SSB plasmid structure and methods of using the structure and system to identify modulators of DDR activity for SSB repair. The present disclosure also provides methods for modulating the defined SSB signaling as well as methods of screening for one or more modulators of SSB mediated DDR activity, and methods of inhibiting SSB repair.

Embodiments of a site-specific, single-strand break (SSB) plasmid structure of the present disclosure include an engineered plasmid, where the plasmid is a double-stranded, circular plasmid having an inner (−) and outer (+) strand, the engineered plasmid genetically modified to have a single recognition site for a specific restriction enzyme, where the single restriction site is located on the +strand of the plasmid, such that contacting the plasmid with the specific restriction enzyme results in a single nick in5 the +strand only.

Methods of making a site-specific, single-strand break (SSB) plasmid structure of the present disclosure are provided in the present disclosure. Embodiments of such methods can include providing a genetically engineered plasmid having a single recognition site for a specific restriction enzyme located on the outer (+) strand of the plasmid DNA and contacting the plasmid with the specific restriction enzyme to generate a single-strand break in the +strand of the plasmid to produce a SSB plasmid structure.

Embodiments of a cell-free single-strand break (SSB) repair and signaling system of the present disclosure can include an engineered site-specific, SSB plasmid structure comprising a single nick in a double-stranded, circular plasmid having an inner (−) and outer (+) strand, wherein the nick is located at a single restriction site in the + strand of the plasmid, and a high-speed supernatant (HSS) from Xenopus egg extracts.

The present disclosure also provides methods for identifying modulators of DNA damage response (DDR) activity for single-strand break (SSB) repair. Embodiments of such methods include providing a composition including a plurality of engineered site-specific, SSB plasmid structures, each having a single nick in a double-stranded, circular plasmid having an inner (−) and outer (+) strand, where the nick is located at a single restriction site in the + strand of the plasmid; and providing a high-speed supernatant (HSS) from Xenopus egg extract, where incubating the engineered site-specific, SSB plasmid structure in the HSS results in one or more SSB DNA damage response (DDR) activities; combining the engineered site-specific, SSB plasmid structure with the HSS and a test compound to make a test mixture; and detecting SSB DDR activity.

Systems for high-throughput identification of small-molecule modulators of DNA damage response (DDR) activity for single-strand break (SSB) repair are provided in the present disclosure. Embodiments of these systems can include an array with a plurality of spots, each spot including: a composition comprising a plurality of engineered site-specific, SSB plasmid structures, each having a single nick in a double-stranded, circular plasmid having an inner (−) and outer (+) strand, where the nick is located at a single restriction site in the +strand of the plasmid; and a high-speed supernatant (HSS) from Xenopus egg extracts. In embodiments, at least a portion of the spots on the array are test spots and wherein each test spot independently includes a different test compound from a library of small-molecules and a detection substrate capable of producing a detectable signal upon occurrence of an SSB DDR activity, where a reduced or increased SSB DDR activity compared to the SSB DDR activity in the absence of the test compound indicates that the test compound modulates SSB DDR activity.

In embodiments, the present disclosure provides methods of inhibiting single-strand break (SSB) repair, such methods including contacting a composition comprising DNA molecules, wherein at least a portion of the DNA molecules have single-strand breaks, with an effective amount of a small molecule inhibitor 3-Hydroxy-9β, 13α-dimethyl-2-oxo-24,25,26-trinoroleana-1(10),3,5,7-tetraen-29-oic acid (Celastrol).

Methods of the present disclosure also include methods for actively inhibiting single-strand break (SSB) repair in at least one cell. In embodiments, such methods can include the step of contacting at least one cell with an effective amount of 3-Hydroxy-9β, 13α-dimethyl-2-oxo-24,25,26-trinoroleana-1(10),3,5,7-tetraen-29-oic acid.

In embodiments, the present disclosure also includes a kit comprising a site-specific, single-strand break (SSB) plasmid structure of the present disclosure, and one or more of: (a) a high-speed supernatant (HSS) from Xenopus egg extract; (b) a detection substrate for detecting SSB DDR activity; or (d) instructions for identifying modulators of DNA damage response (DDR) activity for single-strand break (SSB) repair.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 is a schematic diagram illustrating an embodiment of a defined site-specific SSB plasmid structure of the present disclosure, and the generation of both the site-specific SSB plasmid structures and a corresponding DSB structure. In the illustrated embodiment, pUC19 plasmid (SEQ ID NO: 1) is mutated to produce engineered site-specific SSB plasmid pS (SEQ ID NO: 2), having a defined SSB location located in a portion of the plasmid (SEQ ID NO: 3).

FIG. 2 is a schematic drawing illustrating the preparation and function of the cell-free SSB/HSS system of the present disclosure for demonstrating SSB repair and signaling.

FIGS. 3A-3B are schematic diagrams of the production of LSS, HSS, and NPE (FIG. 3A) and the use of the compounds in systems for analysis of DNA replication, DNA damage repair, and DNA damage response (DDR) processes (FIG. 3B).

FIG. 4 is a schematic illustration of a model of the molecular mechanism of APE2-mediated ATR-Chk1 DDR pathway induced by a defined SSB plasmid structure of the present disclosure.

FIG. 5 is a schematic illustration of an embodiment of a system of the present disclosure for high-throughput screening of compound libraries for small-molecule modulators of DDR activity for SSB repair.

FIGS. 6A-6D illustrate verification and analysis of the defined DNA structures. FIG. 6A is an image verifying the defined SSB structure on agarose gel (Ethidium bromide staining). CTL plasmid was added to HSS for different time. Then DNA samples were isolated and treated with or without Sbfl, and analyzed on agarose gel (Ethidium bromide staining) as shown in FIG. 6B. For FIG. 6C, the SSB plasmid was added to HSS with or without VE-822 for different time as indicated. SSB repair products were isolated and examined on agarose gel (Ethidium bromide staining). FIG. 6D is a graph quantifying SSB repair capacity (circular/(circular+nicked)×100) with or without VE-822 treatment in the HSS system from FIG. 6C.

FIGS. 7A-7C illustrate repair of an embodiment of a site-specific SSB plasmid structure of the present disclosure. FIG. 7A is an image of an agarose gel electrophoresis showing the gradual repair of the SSB structure in an HSS system (intermediate products were isolated at different time points, and treated by Sbfl). FIG. 7B is an image of an agarose gel electrophoresis showing CTL or SSB plasmid in HSS supplemented with [³²P-α]-dATP, for a 30-min incubation. Then NPE was added for continuous incubation for different time as indicated and samples were examined on agarose gel. FIG. 7C is a graph illustrating quantification of DNA synthesis of CTL or SSB plasmid in the HSS/NPE system shown in FIG. 7B.

FIGS. 8A-8G illustrate that the ATR-Chk1 DNA damage response pathway is induced by the defined SSB structure in the HSS system. CTL or SSB plasmid was added to HSS at different concentrations as indicated, and incubated for 30 minutes. Extracts were examined via immunoblotting analysis for Chk1 phosphorylation (i.e., Chk1 P-Ser344) and total Chk1 (FIG. 8A). CTL or SSB plasmid was added to HSS at a final concentration of 75 ng/μL. After different time of incubation at room temperature, the extracts were examined via immunoblotting analysis (FIG. 8B). ATR inhibitor VE-822, ATM inhibitor KU55933, DNA-PK inhibitor NU7441, or recombinant geminin was added to HSS supplemented with CTL or SSB plasmid at a final concentration of 75 ng/4 for 30 minutes. Extracts were examined via immunoblotting analysis as indicated, and results are shown in FIGS. 8C-8E. Geminin or roscovitine was added to HSS supplemented with sperm chromatin and hydrogen peroxide. After a 45-min incubation, extracts were examined via immunoblotting analysis as indicated (FIG. 8F). CTL, SSB, or DSB plasmid was added to HSS at different concentrations as indicated. Samples were examined via immunoblotting analysis (FIG. 8G).

FIGS. 9A-9C illustrate ATR-Chk1 DDR pathway is triggered by SSB plasmid in Xenopus HSS and NPE systems. CTL or SSB plasmid was added to HSS with the presence or absence of VE-822. After 30-min incubation, Chk1 phosphorylation, RPA32 phosphorylation, and Rad17 phosphorylation were examined via immunoblotting analysis as indicated in the image in FIG. 9A. CTL or SSB plasmid was added to NPE with the presence or absence of VE-822 (ATR inhibitor) or Tautomycin (phosphatase inhibitor). Samples were examined via immunoblotting analysis as illustrated in FIG. 9B. For FIG. 9C, CTL or SSB plasmid was added to mock- or Pol alpha-depleted HSS. After 30-min incubation, samples were analyzed via immunoblotting analysis, as illustrated.

FIGS. 10A-10G illustrate the role of APE2 in checkpoint signaling from the defined SSB structure in the HSS system. CTL or SSB plasmid was added to mock-, ATRIP-, TopBP1-, Rad9-, or Claspin-depleted HSS, respectively. Extracts were examined via immunoblotting analysis in FIGS. 10A-10D. For FIG. 10E, CTL or SSB plasmid was added to mock- or XRCC1-depleted HSS at a concentration of 75ng/μL for 30 minutes. Extracts were examined via immunoblotting analysis, as indicated. PARP1 specific inhibitor (4-Amino-1,8-naphthalimide, 0.1 mM) was added to HSS supplemented with CTL or SSB plasmid. Extracts were examined via immunoblotting analysis (FIG. 10F). In FIG. 10G, recombinant Myc-APE2 was added to APE2-depleted HSS supplemented with CTL or SSB plasmid. Extracts were examined via immunoblotting analysis. “Endo. APE2” represents endogenous APE2.

FIGS. 11A-11B illustrate that hydrogen peroxide induces Chk1 phosphorylation and RPA32 phosphorylation in an ATR-dependent manner in human U2OS cells. FIG. 11A is a digital image of immunoblotting analysis of asynchronized U2OS cells treated with H₂O₂ and/or VE-822. Cell lysates were analyzed via immunoblotting analysis as indicated. G1 synchronized U2OS cells were treated with H₂O₂ and/or VE-822. Cell lysates were analyzed via immunoblotting analysis as indicated and illustrated in FIG. 11B.

FIGS. 12A-12E illustrate that APE2 Zf-GRF interacts with PCNA as the second mode of APE2-PCNA interaction. FIG. 12A is a schematic diagram of APE2 Zf-GRF region and the IDOL and CTM regions of PCNA showing 2 modes of interaction. FIG. 12B illustrates GST pulldown assays with GST, GST-APE2, and GST-APE2-ZF from HSS. The input and pulldown samples were examined via immunoblotting analysis. FIG. 12C illustrates GST pulldown assays with GST or GST-APE2-ZF as well as WT/mutant His-tagged PCNA (e.g., LI PCNA, PK PCNA, or LIPK PCNA) in an interaction buffer. The input and pulldown samples were examined via immunoblotting analysis. FIG. 12D illustrates GST pulldown assays with GST or WT/mutant GST-APE2-ZF (i.e., G483A-R484A, F486A-Y487A, or C470A) as well as WT His-tagged PCNA in an interaction buffer. The input and pulldown samples were examined via immunoblotting analysis. FIG. 12E illustrates Biotin-coupled ssDNA (80 nt) was coupled to streptavidin dynabeads and utilized for protein-DNA interaction assays with GST or WT/mutant GST-APE2-ZF (i.e., G483A-R484A, F486A-Y487A, or C470A) in an interaction buffer.

FIGS. 13A-13E illustrate that APE2 Zf-GRF interacts with PCNA and ssDNA for SSB signaling. FIG. 13A illustrates GST pulldown assays with GST, GST-APE2, and GST-APE2-ZF as well as His-tagged WT PCNA in a buffer. The input and pulldown samples were examined via immunoblotting analysis. * represents nonspecific band. FIG. 13B illustrates GST-pulldown assays with GST, WT or R502E GST-APE2-ZF as well as WT PCNA in a buffer. The input and pulldown samples were examined via immunoblotting analysis. Biotin-coupled ssDNA (80 nt) was coupled to streptavidin dynabeads and utilized for protein interaction assays with WT or R502E GST-APE2 in an interaction buffer. The bead-bound and input samples were analyzed via immunoblotting analysis in FIG. 13C. WT or G483A-R484A Myc-APE2 was added to APE2-depleted HSS, which was supplemented with CTL or SSB plasmid, and samples were analyzed via immunoblotting analysis in FIG. 13D. For FIG. 13E, WT or G483A-R484A Myc-APE2 was added to APE2-depleted LSS, which was supplemented with sperm chromatin and hydrogen peroxide. Samples were analyzed via immunoblotting analysis. * represents a non-specific band in LSS overlaps with Myc-APE2.

FIGS. 14A-14D illustrate APE2 Zf-GRF-PCNA interaction promotes SSB end resection, the assembly of a checkpoint protein complex onto SSB plasmid, and Chk1 phosphorylation in the HSS system. CTL or SSB plasmid was added to mock- or APE2-depleted HSS, which was supplemented with WT or C470A Myc-APE2. DNA-bound fractions and total extract samples were examined via immunoblotting analysis as indicated (FIG. 14A). “Endo. APE2” represents endogenous APE2. FAM-labeled dsDNA with a site specific SSB (designed as FAM-SSB) was added to HSS for different time as indicated. Then samples were examined via TBE-Urea gel and visualized via Typhoon imager for FIG. 14B. “Marker” represents four FAM-labeled different-length ssDNA. FIG. 14C illustrates the length dependence of ssDNA for the recruitment of ATR-ATRIP complex and RPA to ssDNA in the HSS. Streptavidin Dynabeads coupled with different length of Biotin-coupled ssDNA (i.e., 0, 10, 20, 40, 60, or 80 nt) were added to HSS. After incubation, the Biotin-ssDNA bead-bound fractions were isolated from HSS. The Input and bead-bound fractions were examined via immunoblotting analysis as shown in FIG. 14C. The FAM-SSB substrate was added to mock- or APE2-depleted HSS, which was supplemented with WT or C470A Myc-APE2. DNA structures were examined via TBE-Urea gel and visualized via Typhoon imager (FIG. 14D, Top). Samples were also analyzed via immunoblotting analysis as indicated. “Endo. APE2” represents endogenous APE2 (FIG. 14D, Bottom).

FIGS. 15A-15C illustrate APE2 exonuclease activity in 3′-5′ SSB end resection, checkpoint protein complex assembly, and SSB-induced Chk1 phosphorylation in the HSS system. For FIG. 15A, the FAM-SSB substrate was treated with increased concentrations of recombinant GST-APE1 (e.g., 0.05, 0.5, 5, and 25 pmol/μL). Samples were analyzed on TBE-Urea gel and visualized via Typhoon imager as illustrated in FIG. 15A. In vitro analysis of exonuclease activity of WT, E34A, or D273A GST-APE2 with or without WT His-tagged PCNA using the FAM-gapped DNA substrate is illustrated in FIG. 15B. For FIG. 15C, WT, E34A, or D273A Myc-APE2 was added back to APE2-depleted HSS, which was supplemented with SSB plasmid. After 30-min incubation, DNA-bound and total extracts were analyzed via immunoblotting analysis as indicated.

FIGS. 16A-16B illustrate exonuclease activities of APE2 with purified proteins in vitro. FIG. 16A illustrates in vitro analysis of exonuclease activity of GST-APE2 with the presence or absence of WT or mutant His-tagged PCNA using the FAM-labeled gapped dsDNA structure. FIG. 16B illustrates in vitro analysis of exonuclease activity of WT and mutant GST-APE2 with the presence or absence of WT his-tagged PCNA using the FAM-labeled gapped dsDNA structure.

FIGS. 17A-17B illustrate DNA binding analysis of APE2 in vitro. FIG. 17A illustrates in vitro protein-DNA interaction assays for GST and GST-APE2 with the presence or absence of WT His-tagged PCNA using streptavidin dynabeads coupled with or without Biotin-gapped dsDNA. * represents the nonspecific bands. FIG. 17B illustrates in vitro protein-DNA interaction assays for GST-APE2 with the presence of WT/mutant His-tagged PCNA using streptavidin dynabeads coupled with Biotin-gapped dsDNA. Samples were examined via immunoblotting analysis as indicated.

FIGS. 18A-D illustrate the chemical structure and inhibitory action of a small molecule, 3-Hydroxy-9β, 13α-dimethyl-2-oxo-24,25,26-trinoroleana-1(10),3,5,7-tetraen-29-oic acid (Celastrol) (PubChem CID 122724), on APE2 mediated SSB signaling and repair. FIG. 18A is a schematic illustration of Celestrol inhibition of APE2-DNA interaction. The addition of Celastrol inhibits SSB-induced Chk1 phosphorylation in the HSS system (FIG. 18B) and the binding of APE2 Zf-GRF to ssDNA in vitro (FIG. 18C). APE2 promotion of PCNA-mediated end resection of FAM-labeled gapped DNA structure was compromised by Celastrol (FIG. 18D).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification that are incorporated by reference, as noted in the application, are incorporated by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, organic chemistry, biochemistry, genetic engineering, and the like, which are within the skill of the art. Such techniques are explained fully in the literature.

It must be noted that, as used in the specification and the appended embodiments, the singular forms “a,” “an,” and “the” include plural referents unless the context clearly dictates otherwise. Thus, for example, reference to “a support” includes a plurality of supports. In this specification and in the embodiments that follow, reference will be made to a number of terms that shall be defined to have the following meanings unless a contrary intention is apparent.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Definitions

In describing the disclosed subject matter, the following terminology will be used in accordance with the definitions set forth below.

As used herein, “about,” “approximately,” and the like, when used in connection with a numerical variable, generally refers to the value of the variable and to all values of the variable that are within the experimental error (e.g., within the 95% confidence interval for the mean) or within +/−10% of the indicated value, whichever is greater.

The terms “nucleic acid” and “polynucleotide” are terms that generally refer to a string of at least two base-sugar-phosphate combinations. As used herein, the terms include deoxyribonucleic acid (DNA) and ribonucleic acid (RNA) and generally refer to any polyribonucleotide or polydeoxyribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes. Thus, for instance, polynucleotides as used herein refers to, among others, single-and double-stranded DNA, DNA that is a mixture of single-and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid sequence” and “oligonucleotide” also encompasses a nucleic acid and polynucleotide as defined above.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein.

The term also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotides” as that term is intended herein.

A “gene” typically refers to a hereditary unit corresponding to a sequence of DNA that occupies a specific location on a chromosome and that contains the genetic instruction for a characteristic(s) or trait(s) in an organism and its regulatory sequences.

As used herein, “isolated” means removed or separated from the native environment. Therefore, isolated DNA can contain both coding (exon) and noncoding regions (introns) of a nucleotide sequence corresponding to a particular gene. An isolated peptide or protein indicates the protein is separated from its natural environment. Isolated nucleotide sequences and/or proteins are not necessarily purified. For instance, an isolated nucleotide or peptide may be included in a crude cellular extract or they may be subjected to additional purification and separation steps.

With respect to nucleotides, “isolated nucleic acid” refers to a nucleic acid with a structure (a) not identical to that of any naturally occurring nucleic acid or (b) not identical to that of any fragment of a naturally occurring genomic nucleic acid spanning more than three separate genes, and includes DNA, RNA, or derivatives or variants thereof. The term covers, for example but not limited to, (a) a DNA which has the sequence of part of a naturally occurring genomic molecule but is not flanked by at least one of the coding sequences that flank that part of the molecule in the genome of the species in which it naturally occurs; (b) a nucleic acid incorporated into a vector or into the genomic nucleic acid of a prokaryote or eukaryote in a manner such that the resulting molecule is not identical to any vector or naturally occurring genomic DNA; (c) a separate molecule such as a cDNA, a genomic fragment, a fragment produced by polymerase chain reaction (PCR), ligase chain reaction (LCR) or chemical synthesis, or a restriction fragment; (d) a recombinant nucleotide sequence that is part of a hybrid gene, e.g., a gene encoding a fusion protein, and (e) a recombinant nucleotide sequence that is part of a hybrid sequence that is not naturally occurring. Isolated nucleic acid molecules of the present disclosure can include, for example, natural allelic variants as well as nucleic acid molecules modified by nucleotide deletions, insertions, inversions, or substitutions.

It is advantageous for some purposes that a nucleotide sequence is in purified form. The term “purified” in reference to nucleic acid represents that the sequence has increased purity relative to the natural environment.

The term “polypeptides” and “protein” include proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, VV), Tyrosine (Tyr, Y), and Valine (Val, V).

“Variant” refers to a polypeptide that differs from a reference polypeptide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0 ±1); glutamate (+3.0 ±1); serine (+0.3); asparagine (+0.2); glutamine (+0.2); glycine (0); proline (−0.5 ±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

As used herein “functional variant” refers to a variant of a protein or polypeptide (e.g., a variant of a CCD enzyme) that can perform the same functions or activities as the original protein or polypeptide, although not necessarily at the same level (e.g., the variant may have enhanced, reduced or changed functionality, so long as it retains the basic function).

“Identity,” as known in the art, is a relationship between two or more polypeptide sequences, as determined by comparing the sequences. In the art, “identity” also refers to the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 48: 443-453, 1970) algorithm (e.g., N BLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the term “engineered” indicates that the engineered object is created and/or altered by human intervention. An engineered object may include naturally derived substances, but the object itself is altered in some way by human intervention and design. For instance, an “engineered” or “genetically engineered” plasmid refers to a plasmid that has been altered in some way (e.g., by genetic modification) by human intervention.

The term “expression” as used herein describes the process undergone by a structural gene to produce a polypeptide. It is a combination of transcription and translation. Expression generally refers to the “expression” of a nucleic acid to produce a polypeptide, but it is also generally acceptable to refer to “expression” of a polypeptide, indicating that the polypeptide is being produced via expression of the corresponding nucleic acid.

As used herein, the term “over-expression” and “up-regulation” refers to the expression of a nucleic acid encoding a polypeptide (e.g., a gene) in a genetically modified cell or cell-free system at higher levels (therefore producing an increased amount of the polypeptide encoded by the gene) than the “wild type” cell or system (e.g., a substantially equivalent cell or system that is not transfected with the gene) under substantially similar conditions. Thus, to over-express or increase expression of a target nucleic acid refers to increasing or inducing the production of the target polypeptide encoded by the nucleic acid, which may be done by a variety of approaches, such as increasing the number of genes encoding for the polypeptide, increasing the transcription of the gene (such as by placing the gene under the control of a constitutive promoter), or increasing the translation of the gene, or a combination of these and/or other approaches. Conversely, “under-expression” and “down-regulation” refers to expression of a polynucleotide (e.g., a gene) at lower levels (producing a decreased amount of the polypeptide encoded by the polynucleotide) than in a “wild type” cell or cell free system. As with over-expression, under-expression can occur at different points in the expression pathway, such as by decreasing the number of gene copies encoding for the polypeptide, inhibiting (e.g., decreasing or preventing) transcription and/or translation of the gene (e.g., by the use of antisense nucleotides, suppressors, knockouts, antagonists, etc.), or a combination of such approaches.

As used herein, the term “increase”, with respect to an activity, process, status, etc., refers to a measurably greater occurrence of such activity/process/status under certain circumstances and/or environments, as compared to a comparative circumstance or environment. Similarly, the term “decrease,” with respect to an activity, process, status, etc., refers to a measurably lesser occurrence of such activity/process/status in a certain circumstance or environment, as compared to a comparative circumstance or environment. For example, an increase in phosphorylation of a particular target peptide in a particular circumstance (e.g., in the presence of a particular test compound) exists when there is a greater occurrence of phosphorylation of that target peptide under the particular circumstances as compared to a control circumstance (e.g., the absence of the particular test compound).

The term “plasmid” as used herein refers to a non-chromosomal, double-stranded DNA sequence including an intact “replicon” such that the plasmid is replicated in a host cell. Plasmids can be linear or circular. Circular plasmids can be described as having an inner and outer strand. The outer strand is referred to herein as the “+ strand,” and the inner strand as the “−strand.”

As used herein, the term “expression system” includes a biologic system (e.g., a cell based system) used to express a polynucleotide to produce a protein. Such systems generally employ a plasmid or vector including the polynucleotide of interest, where the plasmid of expression vector is constructed with various elements (e.g., promoters, selectable markers, etc.) to enable expression of the protein product from the polynucleotide. Expression systems use the host system/host cell transcription and translation mechanisms to express the product protein. Common expression systems include, but are not limited to, bacterial expression systems (e.g., E. coli), yeast expression systems, viral expression systems, animal expression systems, and plant expression systems.

As used herein, the term “promoter” or “promoter region” includes all sequences capable of driving transcription of a coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

The term “operably linked” indicates that the regulatory sequences necessary for expression of the coding sequences of a nucleic acid are placed in the nucleic acid molecule in the appropriate positions relative to the coding sequence so as to effect expression of the coding sequence. This same terminology is sometimes applied to the arrangement of coding sequences and transcription control elements (e.g. promoters, enhancers, and termination elements), and/or selectable markers in an expression vector.

The terms “native,” “wild type”, or “unmodified” in reference to an organism (e.g., plant or cell), polypeptide, protein or enzyme, are used herein to provide a reference point for a variant/mutant of an organism, polypeptide, protein, or enzyme prior to its mutation and/or modification (whether the mutation and/or modification occurred naturally or by human design). Typically, the unmodified, native, or wild type organism, polypeptide, protein, or enzyme has an amino acid sequence that corresponds substantially or completely to the amino acid sequence of the polypeptide, protein, or enzyme as it typically/predominantly occurs in nature.

As used herein, the term “library” refers to a collection of items (e.g., group of sDNA sequences, peptides, group of small molecule chemical compounds, group of cells, group of organisms, etc.), where most of the individual items in the library differ from every other item (or substantially every other item; some small percentage of repeats may be unavoidable) in some aspect.

The term “detectable” refers to the ability to perceive or distinguish a signal over a background signal. “Detecting” refers to the act of determining the presence of and recognizing a target or the occurrence of an event by perceiving a signal that indicates the presence of a target or occurrence of an event, where the signal is capable of being perceived over a background signal. As used herein a “detection substrate” is a substrate that, when acted upon, produces a detectable signal.

The term “phosphorylatable” refers to a target peptide that is capable of being phosphorylated (e.g., having a phosphoryl group coupled to it) by an enzyme, typically a kinase). Phosphorylation/dephosphorylation typically leads to activation or deactivation of many proteins, thereby regulating function. Phosphorylation can occur on several amino acid side chains, such as, serine, threonine, and tyrosine, when those amino acid residues are in a conformation such that they are accessible to a kinase. In embodiments, a “phosphorylatable peptide” refers to a peptide that is phosphorylatable (e.g., by inclusion of a phosphorylatable amino acid residue). Conversely, a “non-phosphorylatable peptide” refers to a peptide that cannot be phosphorylated (e.g., by lacking an exposed phosphorylatable amino acid). For instance, a peptide described below having SEQ ID NO: 4 is phosphorylatable at the serine residue at aa 10, whereas the peptide described below having SEQ ID NO: 5 is not phosphorylatable, because in place of the serine, it has an alanine residue at aa 10.

In embodiments, the phosphorylation of a target peptide can serve as a “detection substrate” for producing a “detectable signal” in the methods and systems of the present disclosure.

As used herein, the term “single-strand break DNA damage response activity” (SSB DDR activity) refers to the occurrence of one or more activates related to a series of sub-cellular events in a DNA response and repair process initiated by the occurrence and recognition of a single-strand break in a double stranded DNA molecule, which events include, but are not limited to, recognition of a single-strand break, signaling related to the single-strand break, recruitment and activation of various compounds involved in the DDR pathway, and culminating with the repair of the single-strand break. Examples of events that can occur during this process and thus represent one or more “SSB DDR activities” include, but are not limited to recruitment of APE2 to the site of a SSB, interaction of the PIP (PCNA interaction protein) box of APE2 with the PCNA-IDCL (interdomain connector loop), interaction of the APE2 zinc-finger motif (Zf-GRF) with the PCNA CTM and/or the portion of ssDNA at the site of the SSB, APE2 exonuclease activity, binding of RPA to the ssDNA, recruitment of ATR and ATRIP to the region of ssDNA, phosphorylation of Chk1 by ATR, interaction of the 911 complex and/or TopBP1 with the region of ssDNA to repair the SSB and restore the dsDNA. In embodiments, the ATR phosphorylation of Chk1 via APE2 recruitment and activation is used as a SSB DDR activity that indicates activation of the SSB DDR pathway.

By “administration” is meant introducing a compound of the present disclosure into a subject; it may also refer to the act of providing a composition of the present disclosure to a subject (e.g., by prescribing).

The term “effective amount” refers to that amount of the compound being administered which will produce a reaction that is distinct from a reaction that would occur in the absence of the compound. The term “therapeutically effective amount” as used herein refers to that amount of the compound being administered which will relieve or prevent to some extent one or more of the symptoms of the condition to be treated. In reference to conditions/diseases that can be directly treated with a composition of the disclosure, a therapeutically effective amount refers to that amount which has the effect of preventing the condition/disease from occurring in an animal that may be predisposed to the disease but does not yet experience or exhibit symptoms of the condition/disease (prophylactic treatment), alleviation of symptoms of the condition/disease, diminishment of extent of the condition/disease, stabilization (e.g., not worsening) of the condition/disease, preventing the spread of condition/disease, delaying or slowing of the condition/disease progression, amelioration or palliation of the condition/disease state, and combinations thereof.

### Description:

Embodiments of the present disclosure encompass structures, systems, and methods for modeling and analysis of single-strand break (SSB) signaling and repair in a cell-free system, methods of making the SSB structures and systems, methods and systems for identifying modulators of DNA damage response (DDR) activity for SSB repair, and methods of inhibiting SSB repair.

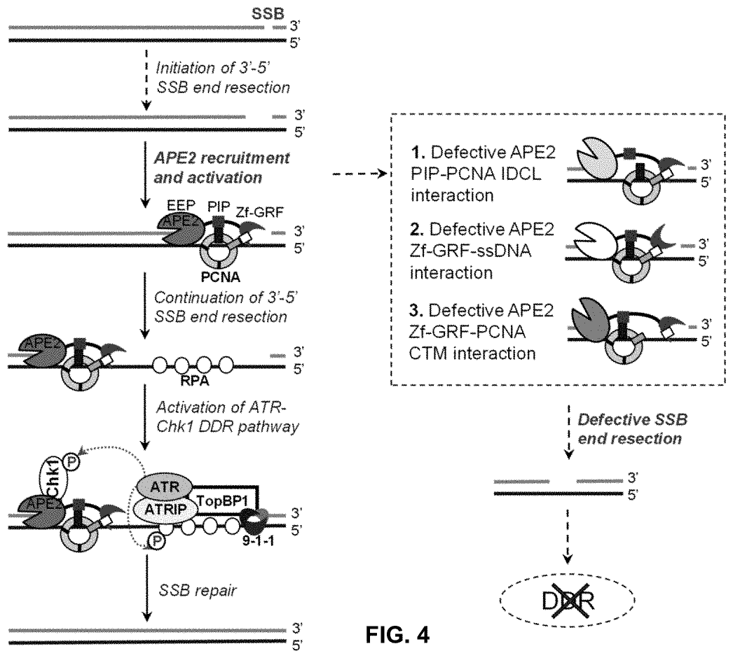

Some mechanisms and principles of cellular response to DNA damage (single or double strand breaks, etc.) have been studied. It is generally accepted that cellular response to DNA damage and replication stress is mainly coordinated by ATR-Chk1 and ATM-Chk2 DNA damage response (DDR) pathways (Bartek et al., 2004; Branzei and Foiani, 2010; Harper and Elledge, 2007; Harrison and Haber, 2006; Su, 2006). Mechanisms to activate the ATR-Chk1 DDR pathway include, but are not necessarily limited to stalled DNA replication forks, UV-damage, DSBs, or oxidative DNA damage (Ciccia and Elledge, 2010; Cimprich and Cortez, 2008). Full ATR activation requires several mediator proteins, such as ATRIP, TopBP1, MDC1, and the 9-1-1 (Rad9-Rad1-Hus1) complex (see, e.g., FIG. 4, described in Example 1, below). In response to stalled DNA replication forks or UV-damage, ATR can be activated by primed single-stranded DNA (ssDNA) from functional uncoupling of MCM helicase and DNA polymerase activities (Byun et al., 2005). For DSBs (double-strand breaks), ATR can be activated after the 5′-3′ end resection of DSBs (Sartori et al., 2007; Shiotani and Zou, 2009). Also, in response to oxidative stress, ATR is activated through APE2-mediated DNA end resection of oxidative DNA damage in the 3′-5′ direction (Willis et al., 2013).

It is also generally accepted that RPA-coated long stretch of ssDNA serves as platform to recruit ATR and TopBP1 to sites of DNA damage, although the 9-1-1 complex prefers the 5′-recessed ssDNA/dsDNA junctions (Acevedo et al., 2016; Ellison and Stillman, 2003; Marechal and Zou, 2015; Zou and Elledge, 2003). Activated ATR kinase phosphorylates a variety of substrates including Chk1 to regulate cell cycle progress, activate transcription, and promote DNA repair (Matsuoka et al., 2007). Upon phosphorylation at the Ser345 or Ser317 residue of Chk1, Chk1 kinase will be fully activated, and Chk1 phosphorylation is often utilized as an indicator of ATR activation (Chen and Sanchez, 2004; Guo et al., 2000; Zhao and Piwnica-Worms, 2001). A two-step model for DNA end resection at DSB sites has been proposed through MRN (Mre11-Rad50-Nbs1) complex or CtIP/Sae2, and Exo1 or DNA2/Sgs1 (19). Furthermore, ATM can be activated through a disulfide bond formation and conformation change in oxidative stress in a DNA-independent manner (20,21).

Although both SSBs and DSBs activate the DDR pathways, the repair signaling and mechanisms for SSBs are probably less understood. SSBs lead to genome instability and have been associated with various pathologies, and although ATM can be activated by presumptive unrepaired SSBs in XRCC1-dificient cells, it remains unknown how exactly unrepaired SSBs activate ATM DDR pathway (21). As mentioned above, the lack of a defined SSB experimental system and the inability to distinguish SSBs from DSBs has hindered further understanding of SSB recognition and repair pathways.

Whereas APE1 is the major AP endonuclease (26), APE2 (APEX2, APN2) has strong 3′-5′ exonuclease and 3′-phosphodiesterase activities but weak AP endonuclease activity (27). APE2 is involved in normal B cell development and recovery from chemotherapy drug-induced DNA damage (28). The interdomain connector loop (IDOL) of PCNA associates with the PIP (PCNA interaction protein) box of its interacting proteins (29). The PIP box of APE2 is important for PCNA association (24,30,31). Importantly, APE2 is a key player in PCNA-dependent repair of hydrogen peroxide-induced oxidative DNA damage (30,31). It has been demonstrated that ATR-Chk1 DDR pathway is activated by hydrogen peroxide-induced oxidative stress in Xenopus, and that APE2 is important for the oxidative stress-induced ATR-Chk1 checkpoint signaling (24). Notably, the examples below demonstrate that a zinc-finger motif (referred to herein as Zf-GRF) in APE2's C-terminus associates with ssDNA, but not dsDNA, and that APE2 Zf-GRF facilitates 3′-5′ end resection of oxidative DNA damage to promote ATR-Chk1 DDR pathway (32).

As a cell-free experimental system from eggs of the African clawed frogs, Xenopus egg extracts have been widely used in studies of chromosome metabolisms, and findings from Xenopus system can be validated in mammalian cell lines (Costanzo and Gautier, 2004; Deming and Kornbluth, 2006; Kumagai and Dunphy, 2000; Lebofsky et al., 2009; Lupardus et al., 2002; Michael et al., 2000; Philpott and Yew, 2008; Raschle et al., 2008; Tutter and Walter, 2006; Willis et al., 2012). Three different types of Xenopus egg extracts have been widely used: low-speed supernatant (LSS), high-speed supernatant (HSS), and nucleoplasmic extracts (NPE), discussed in greater detail below (Cupello et al., 2016; Walter et al., 1998). It has been demonstrated in recent studies that APE2 is required for the ATR-Chk1 checkpoint activation in response to oxidative stress-derived SSBs in Xenopus LSS system (Willis et al., 2013; Yan et al., 2014).

Based on the above studies, it was believed that a zinc-finger motif (designated as Zf-GRF) in APE2's C-terminus may associate with ssDNA and 3′-recessed ssDNA/dsDNA junction, but not dsDNA, and that APE2 Zf-GRF facilitates its 3′-5′ end resection of oxidative DNA damage to promote ATR-Chk1 DDR pathway in the LSS system (Wallace et al., 2017). However, this could not be confirmed in current systems, nor did a system exist for the exclusive study of SSB signaling and repair mechanisms, or identification of specific modulators of SSB DDR activities. The defined SSB structures and systems provided in embodiments of the present disclosure were developed for further investigation of the role of APE2 and other proteins in SSB-specific signaling and repair and for use in systems for the identification of modulators of SSB DDR pathway and activities. As discussed in greater detail in the Examples below, development and use of the defined SSB plasmid structure and systems of the present disclosure demonstrated that an ATR-dependent but replication-independent DDR pathway is activated by the defined SSB structure in the Xenopus HSS system. The Examples demonstrate that SSB signaling implements APE2 and canonical checkpoint proteins including ATR, ATRIP, TopBP1, Rad9, and Claspin. Surprisingly, it was found that APE2's Zf-GRF associates with PCNA through its C-terminus. The present disclosure also demonstrates that the distinct APE2-PCNA interaction plays a role for the 3′-5′ SSB end resection and SSB signaling in a eukaryotic system. In addition, the examples provide evidence that the SSB-induced ATR activation is important for SSB repair and that hydrogen peroxide triggers ATR-dependent DDR pathway in human cultured cells. Various embodiments of these structures, systems, and methods of the present disclosure are described below.

Single-Strand Break Plasmid Structures

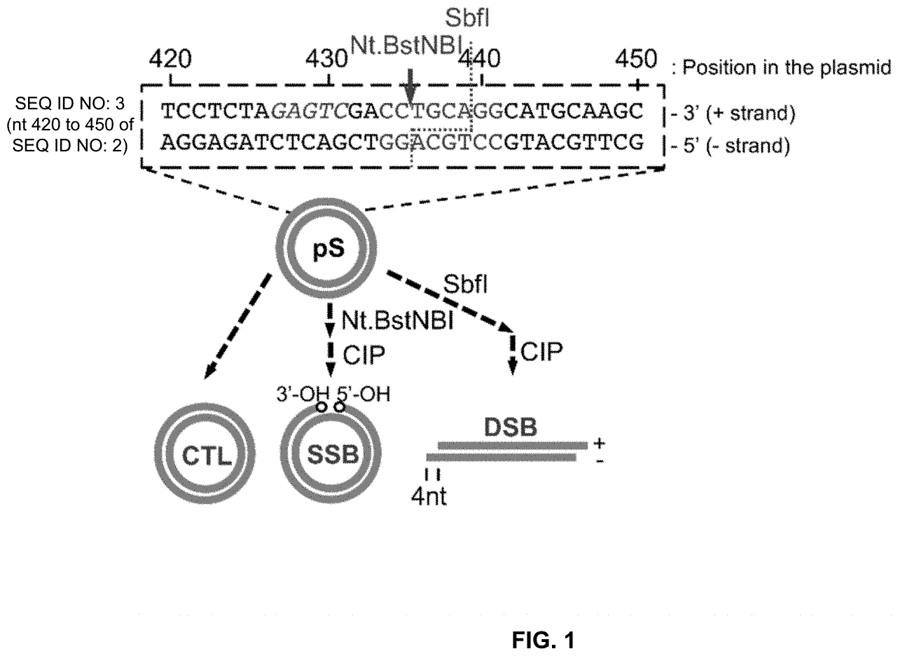

Embodiments of the present disclosure provide site-specific, single-strand break (SSB) plasmid structures, methods of making them and methods of using the site-specific, SSB plasmid structures. Embodiments of the SSB plasmid structures are illustrated in FIG. 1A. The plasmid structures of the present disclosure include an engineered double-stranded, circular plasmid structure. Like typical circular plasmids, the SSB plasmid structures of the present disclosure are circular plasmids having an inner (−) and outer (+) strand. However, the engineered SSB plasmids of the present disclosure have been genetically modified to have a single recognition site for a specific restriction enzyme, wherein the single restriction site is located on the +strand of the plasmid. Thus, where a wild-type or unmodified plasmid may have multiple or zero recognition sites for a particular restriction enzyme, the engineered plasmid structures of the present disclosure have been modified to have only a single recognitions site for that particular restriction enzyme, such that contacting the plasmid with the specific restriction enzyme results in only a single nick in the +strand only, as illustrated in FIG. 1A.

For instance, the unmodified plasmid pUC19 (SEQ ID NO: 1) has four recognition sites for the restriction enzyme Nt.BstNBI, two on the (+) strand and two on the (−) strand. In embodiments of the present disclosure, this plasmid is mutated to produce engineered site-specific SSB plasmid structure pS (SEQ ID NO: 2). The pUC19 plasmid is mutated by removing three of the Nt.BstNBI recognition sites and retain only one recognition site on the +strands (at nt 427-431 of SEQ ID NO: 2, which is within a portion of SEQ ID NO: 2 from nt 420-450, also named SEQ ID NO: 3, herein), as illustrated in FIG. 1A. It will be understood to a skilled artisan that such modifications can be made to many different types of plasmids, and with various restriction sites specific to various restriction enzymes, and such embodiments are intended to be within the scope of the present disclosure. In embodiments, the plasmid is a genetically engineered pUC19 plasmid. In embodiments, the plasmid (pUC19 plasmid or other) is genetically engineered to have a single recognition site for an Nt.BstNBI restriction enzyme on the plasmid +strand. The single recognition site for a specific restriction enzyme enables creation of a single nick in the +strand such that contacting the plasmid with the specific restriction enzyme (e.g., Nt.BstNBI, as shown in FIG. 1A) cuts the +strand only at the one site, resulting in a single nick in the +strand at the location of the single recognition site. In embodiments, the plasmid is a genetically engineered pUC19 plasmid engineered to have a single recognition site for a Nt.BstNBI restriction enzyme in the +strand.

For purposes of comparison of the SSB to a DSB, in embodiments, the SSB plasmid structures of the present disclosure, in addition to the single recognition site for a specific restriction enzyme in the +strand, the pS plasmid also includes a single recognition site for another restriction enzyme such that contacting the plasmid with the other restriction enzyme results in a double-stranded break (DSB) in the plasmid structure, thereby linearizing the plasmid. In embodiments, a SSB plasmid structure of the present disclosure provides a genetically engineered pUC19 plasmid (the genetically engineered pUC19 plasmid is also referred to herein as pS plasmid) with a single recognition site for NT.BstNBI and further comprises a single recognition site for a Sbfl restriction enzyme, such that contacting the plasmid with the Sbfl restriction enzyme results in a double strand break (DSB) in the plasmid, linearizing the plasmid (as shown in FIG. 1A). In embodiments, the Sbfl recognition site is located between residues 434-441 of SEQ ID NO. 1. Thus, in embodiments, the SSB plasmid structure of the present disclosure includes SEQ ID NO: 3 at nt 420-450 of the plasmid, which includes a single recognition site for Nt.BstNBI and Sbfl, and where the plasmid does not include any other recognition sites for these restriction enzymes. Although embodiments of the SSB plasmid structures of the present disclosure are described above with engineered recognition sites for restriction enzymes Nt.BstNBI or Sbfl, in practice, recognition sites for different restriction enzymes can be engineered into different plasmids to generate the SSB plasmid structure of the present disclosure.

Embodiments of the present disclosure also include methods of making the site-specific, single-strand break SSB plasmid structures of the present disclosure. Such embodiments can include genetically engineering a plasmid to have a single recognition site for a specific restriction enzyme located on the outer (+) strand of the plasmid DNA. Methods include providing the genetically engineered plasmid having the single recognition site for a specific restriction enzyme in the (+) strand. The methods then include contacting the plasmid with the specific restriction enzyme (e.g., incubating the plasmid structure with a volume of the restriction enzyme) to generate a single-strand break in the +strand of the plasmid to produce a SSB plasmid structure. As mentioned above, this technique is described in the examples below with respect to the pUC19 plasmid to make the genetically engineered pS plasmid with a single recognition site for the restriction enzyme Nt.BstNBI; however, in practice different plasmid structures and different restriction enzymes/recognition sties can be used. In embodiments, the methods of making the site-specific SSB plasmid structure include contacting the plasmid with a phosphatase to remove a phosphate from a nicked 5′ end of the plasmid DNA at the location of the single-strand break. In embodiments the phosphatase may be contacted with the plasmid simultaneously with the restriction enzyme, or after contacting the plasmid with the specific restriction enzyme. The removal by the phosphatase at the nick site leaves both nicked ends with hydroxyl groups, thereby preventing spontaneous re-ligation of the SSB.

The site-specific SSB plasmid structures of the present disclosure can be included in cell-free, single-strand break (SSB) repair and signaling systems of the present disclosure, and used to identify modulators of DNA damage response (DDR) pathway.

Cell-Free Single-Strand Break (SSB) Repair and Signaling Systems and Kits

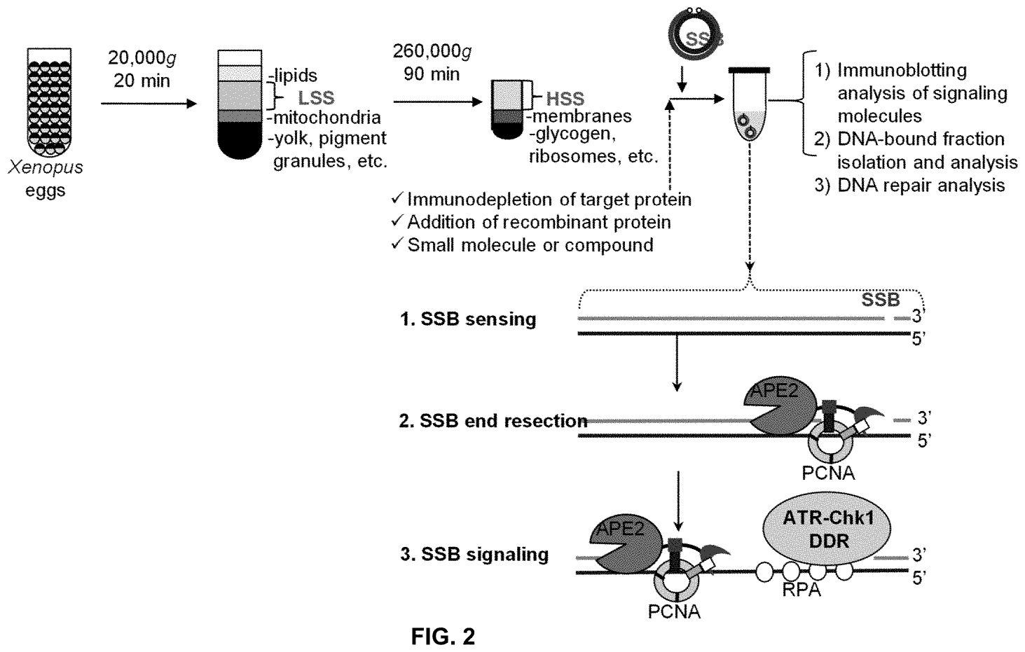

The present disclosure also provides cell-free SSB repair and signaling systems including the engineered site-specific, SSB plasmid structures of the present disclosure and a high-speed supernatant (HSS) from Xenopus egg extracts. As described above, the SSB plasmid structures have a single nick in the +strand of a double-stranded, circular plasmid where the nick is located at a single restriction site in the +strand of the plasmid. The Xenopus egg extract HSS is a composition obtained through specific processing of Xenopus egg extracts, and offers the advantage of being able to observe, manipulate, and study the SSB plasmid structures and SSB DDR activities in a cell-free environment. FIG. 2 is a schematic diagram illustrating aspects the cell-free SSB repair and signaling system of the present disclosure. As shown in FIG. 2, the HSS is obtained from Xenopus eggs (as described in greater detail below), and then the HSS is combined with the SSB plasmid structures to provide the cell-free SSB repair and signaling system of the present disclosure. This system provides for detection and sensing of SSB structures (e.g., detection and confirmation of SSB structures), analysis of SSB DDR activities, such as, but not limited to: SSB end resection (facilitated by recruitment of APE2 and PCNA and interaction of these proteins with the SSB structure), SSB signaling (e.g., coupling of RPA to the SSB structure, recruitment of other proteins involved in SSB DDR activities, including ATR-mediated phosphorylation of Chk1).

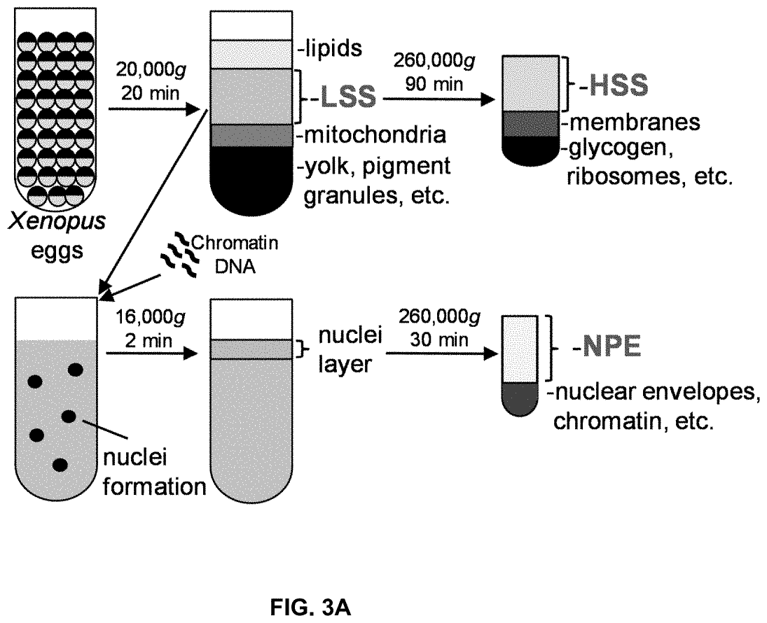

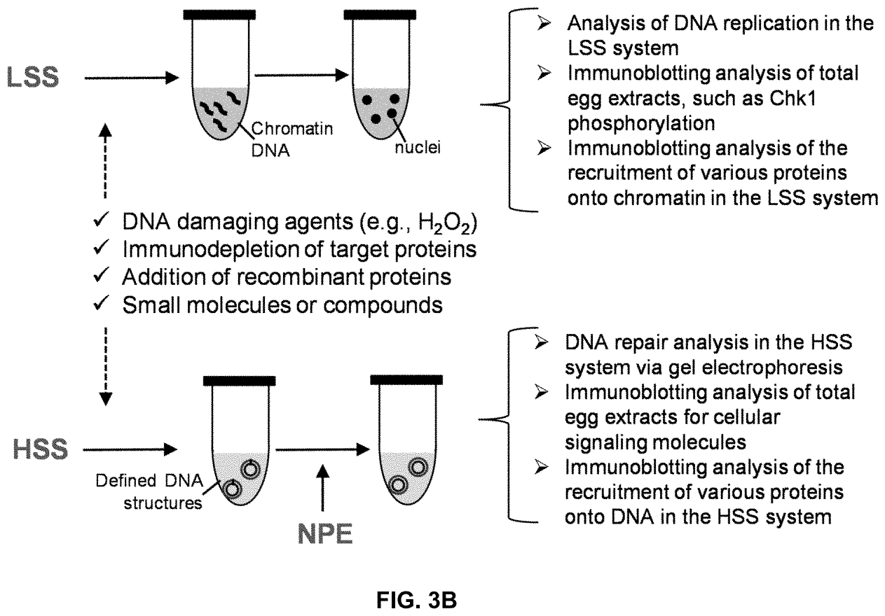

Xenopus egg extracts derived from eggs of African clawed frogs have been utilized in studies of DNA replication, DNA repair, and DNA damage response (DDR) pathways (Costanzo and Gautier, 2004; Karpinka et al., 2015; Kumagai and Dunphy, 2000; Lupardus et al., 2002; Michael et al., 2000; Philpott and Yew, 2008; Raschle et al., 2008; Willis et al., 2013). There are several different types of Xenopus egg extracts: low-speed supernatant (i.e., LSS), high-speed supernatant (i.e., HSS), and nucleoplasmic extracts (i.e., NPE), the production and uses of which are illustrated in FIGS. 3A-3B. Briefly, Xenopus eggs are crushed by centrifugation at low speed (in embodiments, about 18,000 to about 22,000 g, e.g., about 20,000 g) to prepare LSS. Then LSS can be further centrifuged at a high-speed (in embodiments, about 240,000-280,000 g, e.g., about 260,000 g) to prepare HSS. In an LSS system, sperm chromatin can be assembled into nuclei, which are further centrifuged into NPE at a high-speed (in embodiments, about 240,000-280,000 g, e.g., about 260,000 g) (FIG. 3A). The approaches of how these different Xenopus egg extracts are made have been described and reviewed previously (Cupello et al., 2016; Lebofsky et al., 2009, which are hereby incorporated by reference herein).

Thus, in embodiments of the cell-free SSB repair and signaling system of the present disclosure, the HSS is obtained by the following steps: centrifuging Xenopus eggs at a speed of about 18,000-22,000 g for about 20-30min; retaining a low-speed supernatant (LSS) layer; centrifuging the LSS at about 240,000-280,000g, for about 90-120min; and retaining the supernatant layer to produce the HSS. In embodiments the LSS is obtained after centrifuging the eggs at a speed of about 20,000 g for about 20 min. In embodiments, the HSS is obtained after centrifuging the LSS at a speed of about 260,000 g for about 90 min.

LSS, HSS, and NPE can be used for different purposes and analysis. For instance, after being added to the LSS, sperm chromatin DNA or bacteriophage lambda DNA can form nuclear envelope and be replicated in a semi-conservative manner, reconstituting an in vitro cell-free DNA replication system that mimics the in vivo DNA replication program in mammalian cells (Blow and Laskey, 1986; Newport, 1987). When DNA damaging agents are used to stress chromatin DNA in LSS system, immunoblotting analysis of proteins of interest (e.g., Chk1 phosphorylation at Ser 344 and ATM phosphorylation at Ser 1981) can dissect molecular mechanisms of DDR pathways (FIG. 3B). Chromatin bound fractions can be isolated through sucrose cushion and analyzed via immunoblotting analysis (FIG. 3B). Defined DNA structures, such as wild type plasmid DNA or plasmid DNA with an ICL (inter-strand crosslink) at a defined location, such as the SSB plasmid structures of the present disclosure, can initiate pre-replication complex assembly in the HSS, allowing study of SSB signaling and repair via gel electrophoresis, immunoblotting analysis for cellular signaling molecules, immunoblotting analysis of recruitment of various proteins onto the SSB plasmid structure, etc. However, the DNA replication of plasmid DNA can't be elongated without further addition of the NPE, which contain kinase activities of S-CDK (S-phase cyclin-dependent kinase) and DDK (Dbf4-dependent kinase Cdc7-Dbf4) (FIG. 3B). This unique characteristic of the Xenopus HSS/NPE system uncouples DNA replication initiation from replication elongation. Importantly, plasmid DNA with well-defined damage, such as the SSB plasmid structures of the present disclosure described above can be repaired in the HSS system, allowing analysis of the relevant cellular signaling mechanisms related specifically to SSB DDR, as opposed to both SSB and DSB recognition and repair.

Some advantages of the LSS system and the HSS/NPE system are that target proteins can be removed via immunodepletion with specific antibodies and that recombinant wild type or mutant proteins can be added back to depleted egg extracts. Another feature of Xenopus systems is that small molecules (e.g., ATM specific inhibitor KU55933 and ATR specific inhibitor VE-822) can be added to LSS or HSS to certain concentrations allowing analysis of the roles and mechanisms of these small molecules with respect to DDR pathways (see FIG. 3B). In addition, Xenopus egg extracts can be aliquoted, frozen and stored in freezers at −80° C. for multiple experiments. Another advantage of the present system includes that embodiments of the cell-free SSB repair and signaling system of the present disclosure, HSS can be used without addition of NPE (FIG. 2). After incubation of the defined SSB plasmid structure in the HSS (e.g., for about 15 min or more, e.g. 30 min) at room temperature, the DNA-bound fractions can be analyzed, and total extracts for repair or DDR molecules can be determined via immunoblotting analysis.

Thus, in embodiments of the cell-free SSB repair and signaling system of the present disclosure, incubating the engineered site-specific, SSB plasmid structure in the HSS results in one or more DNA damage response (DDR) activities selected from the group consisting of: initiation of DDR processes, recruitment of DDR signaling molecules, formation of DDR protein complexes, and repair of the engineered site-specific, SSB plasmid structure to form an intact circular plasmid. In embodiments, one or more test compounds can be included and/or added to the cell-free SSB repair and signaling systems of the present disclosure. Incubating the engineered site-specific, SSB plasmid structure in the HSS with the test compound allows evaluation of the effect of the test compound on one or more of the DDR activities using the analysis approaches described above, such as immunoblotting analysis of cellular signaling molecules, immunoblotting for recruitment of various DDR proteins, or detection of a DDR event via a detectable signal, such as phosphorylation of a phosphorylatable protein involved in a DDR pathway (or a phosphorylatable peptide derived from such protein, as described in greater detail below).

Embodiments of the present disclosure also include kits including the SSB plasmid structures of the present disclosure described above and one or more of an HSS from Xenopus egg extract, a detection substrate for detecting SSB DDR activity, or instructions for identifying modulators of DDR activity for SSB repair. In embodiments, uch kits can include SSB plasmid structures, HSS, detection substrates, and instructions for identifying modulators.

Methods for Identifying Modulators of DNA Damage Response (DDR) Activity for Single-Strand Break (SSB) Repair

The present disclosure provides methods for identifying modulators of DNA damage response (DDR) activity for single-strand break (SSB) repair using the SSB plasmid structures and cell-free SSB repair and signaling systems of the present disclosure. In embodiments the methods for identifying modulators of DDR activity for SSB repair include providing a composition including a plurality of engineered site-specific, SSB plasmid structures of the present disclosure described above; providing a HSS from Xenopus egg extracts as described above; combining the engineered site-specific, SSB plasmid structure with the HSS and a test compound to make a test mixture; and detecting SSB DDR activity. In embodiments, the method includes screening the test mixture for one or more SSB DDR activities and detecting an SSB DDR activity. Since incubating the engineered site-specific, SSB plasmid structure in the HSS alone results in one or more SSB DDR activities (as described above and described in greater detail in the Examples below), then any changes in the SSB DDR activities (e.g., reduced or increased SSB DDR activity) seen in the presence of the test compound, as compared to the SSB DDR activity in the absence of the test compound, indicate that the test compound modulates an SSB DDR activity.

As described above, in embodiments, SSB DDR activities can be indicated by screening for conditions, such as, but not limited to, the presence of nicked SSB plasmids vs. repaired circular plasmids, the presence or activation (e.g., phosphorylation) of certain cellular signaling molecules, and the recruitment and/or activation of various DDR proteins onto SSB DNA in the HSS system, and the like. See, FIG. 3B and FIG. 4). As described above, these activities can be detected using methodologies known to those of skill in the art, such as, but not limited to, gel electrophoresis to determine form of plasmid DNA (e.g., nicked, circular, linear), immunoblotting analysis of egg extracts for cellular signaling molecules and/or proteins involved in the DDR process, phosphorylation detection of phosphorylatable proteins or peptides involved in the DDR process, and the like.

In embodiments, the methods for identifying modulators of DDR activity for SSB repair of the present disclosure further includes adding a detection substrate to the tests mixture and screening for a detectable signal produced by the detection substrate upon the occurrence of one or more SSB DRR activities. ATR is a kinase capable of phosphorylating its substrates. Activation of ATR is a SSB DDR activity; thus, phosphorylation of an ATR kinase substrate (or a phosphorylatable peptide derived from an ATR kinase substrate) can be used as an indicator of SSB DRR activity. In embodiments, a detection substrate can be a phosphorylatable protein substrate of an ATR kinase or a phosphorylatable peptide derived from a substrate of ATR kinase to the test mixture, and screening for phosphorylation of such protein or peptide. Phosphorylation of a substrate by ATR kinase is a DDR activity. Thus, detecting phosphorylation of the phosphorylatable substrate of ATR kinase or peptide derived therefrom indicates the occurrence of a DDR activity, so a substrate of ATR kinase can act as a detection substrate for indicating occurrence of a SSB DDR activity. In embodiments detecting SSB DDR activity includes detecting phosphorylation of a phosphorylatable peptide derived from a substrate of ATR kinase.

In embodiments, a positive control spot without a test compound has a positive indicator of SSB DDR activity, such as phosphorylated peptide derived from a substrate of ATR kinase. Thus, in embodiments, a positive control produces a detectable phosphorylation signal to indicate SSB DDR Activity. In this manner, if the phosphorylation signal is detected in a test spot and is about the same or increased over the signal in a positive control spot that does not have the test compound, it indicates that the test compound increases or upregulates the DDR activity. If the phosphorylation signal is not detected in a test spot or the signal is decreased over the signal in the positive control spot that does not include the test compound, it indicates that the test compound decreases or suppresses/downregulates the DDR activity. Thus, methods of the present disclosure can also include comparing the SSB DDR activity level (as determined e.g., by detecting phosphorylation of a phosphorylatable detection substrate) in the presence of the test compound to the SSB DDR activity level in the absence of a test compound (e.g., in a control reaction or spot). Modulators of SSB DDR activity may be useful for a variety of reasons, such as cancer treatment, and the like.

One substrate of ATR is Chk1, and phosphorylated Chk1 is also an indicator of activated APE2 (see FIG. 4). Thus, in embodiments, phosphorylation of Chk1 serves as an indicator of SSB DDR activity, and Chk1 or a peptide derived from Chk1 can act as a detection substrate. In embodiments, the SSB DDR activity is selected from the group consisting of: APE2 activation, activation of an ATR complex, or both. In embodiments, APE2 activation, activation of ATR complex, or both are indicated by detecting phosphorylation of Chk1 or a phosphorylatable Chk1-derived peptide. In embodiments, detecting phosphorylation of a phosphorylatable Chk1-derived peptides comprises detecting incorporation of radiolabeled ATP in to the Chk1-derived peptide. In embodiments, the phosphorylatable Chk1-derived peptide is a Chk1 peptide having SEQ ID NO: 4, (LVQGKGISFSQPACPDNML) where phosphorylation occurs at the serine residue at amino acid 10 of SEQ ID NO: 4 (shown in bold).

The methods of identifying modulators of DDR activity for SSB repair of the present disclosure can also include an array with a plurality of spots, where each spot receives the plurality of engineered site-specific, SSB plasmid structures, the high-speed supernatant (HSS) from Xenopus egg extract, and a detection substrate and where a portion of the spots (e.g., test spots) independently receive a test compound. In embodiments, the detection substrate is a phosphorylatable peptide derived from a substrate of ATR kinase, such as described above. Such embodiments can be used in high-throughput systems for screening libraries of compounds for the ability to affect SSB repair activity and DDR pathway.

Systems for High-Throughput Identification of Modulators of DDR Activity for SSB Repair

The present disclosure provides systems for high-throughput identification of small-molecule modulators of DDR for SSB repair that use the methods of identifying modulators of DDR activity described above and the SSB plasmid structures and cell-free SSB repair and signaling systems described above.

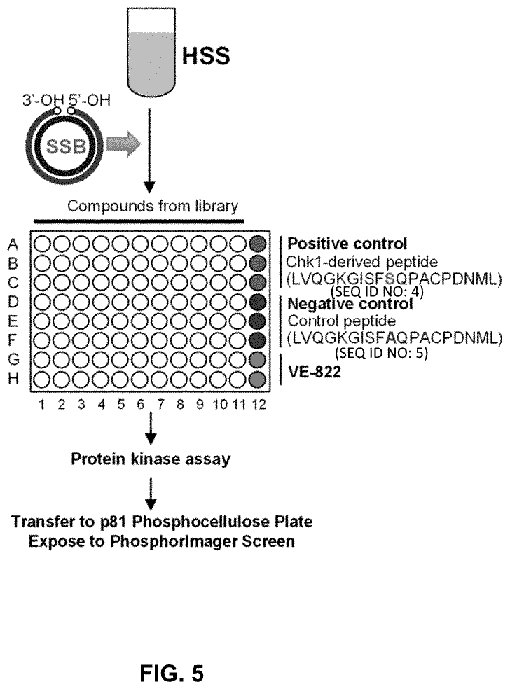

In embodiments, systems for high-throughput identification of small-molecule modulators of DDR for SSB repair of the present disclosure include an array with a plurality of spots, such as illustrated in FIG. 5. Each spot in the array can include a plurality of engineered site-specific, SSB plasmid structures as described above and a HSS from Xenopus egg extracts described above, where incubating the engineered site-specific, SSB plasmid structure in the HSS results in SSB DDR activities. In the high-throughput systems of the present disclosure, a portion of the spots on the array are test spots, where each test spot includes (in addition to the SSB plasmid structure and the HSS) a different test compound and a detection substrate capable of producing a detectable signal upon occurrence of an SSB DDR activity. In an embodiment, the test compounds are from a library of small molecules. Reduced or increased SSB DDR activity, as indicated by the detectable signal of the detection substrate, as compared to the SSB DDR activity in the absence of the test compound indicates that the test compound modulates SSB DDR activity.

In embodiments, such as illustrated in FIG. 5, the detection substrate is a phosphorylatable peptide derived from a substrate of ATR kinase, such as described above, where the detectable signal is phosphorylation of the phosphorylatable peptide, which indicates occurrence of an SSB DDR activity including, but not limited to, APE2 activation, activation of an ATR complex, or both. Since, as described above, Chk1 is phosphorylated by ATR kinase, in embodiments, Chk1 or a phosphorylatable Chk1-derived peptide is the detection substrate, and phosphorylation of Chk1 or the phosphorylatable Chk1-derived peptide is the detectable signal. In embodiments, a positive indicator of SSB DDR compound is phosphorylation of the phosphorylated Chk1-derived peptide. In embodiments, the detection substrate is a phosphorylatable Chk1-derived peptide having SEQ ID NO: 4.

In embodiments, the array also includes at least one positive control spot and at least one negative control spot, such as illustrated in FIG. 5. The at least one positive control spot includes a positive indicator of SSB DDR activity, and the at least one negative control spot includes a negative indicator for a SSB DDR activity. In embodiments, the positive indicator of SSB DDR activity comprises a phosphorylatable Chk1-derived peptide, and the negative indicator of SSB DDR activity comprises a non-phosphorylatable Chk1-derived peptide. Thus, in such embodiments, in the positive control spot, which also includes the SSB plasmid structure of the present disclosure and HSS and does not include a test compound, the Chk1-derived peptide will be phosphorylated, giving a positive indicator of SSB DDR activity. In the negative control spot, which also includes the SSB plasmid structure of the present disclosure and HSS and does not include a test compound, the non-phosphorylatable Chk1-derived peptide is incapable of being phosphorylated, and the lack of phosphorylation provides a negative indicator of SSB DDR activity.

In embodiments, the detection substrate is a phosphorylatable Chk1-derived peptide and the phosphorylation of the phosphorylatable Chk1-derived peptide indicates occurrence of an SSB DDR activity in the test spot and absence or reduced phosphorylation of the phosphorylatable Chk1-derived peptide in the test spot indicates that the test compound suppresses or inhibits an SSB DDR activity. In embodiments, the positive indicator of SSB DDR activity in the positive control spot is the same phosphorylatable Chk1-derived peptide as the detection substrate and the negative indicator of SSB DDR activity in the negative control spot is a non-phosphorylatable Chk1-derived peptide. In such embodiments, phosphorylation of the phosphorylatable detection substrate in a test spot indicates that the test compound has no effect or a positive effect on SSB DDR activity. Increased phosphorylation of the phosphorylatable detection substrate, as compared to the positive control spot indicates that the test compound increases/upregulates a SSB DDR activity. Absence of phosphorylation or reduced phosphorylation of the phosphorylatable Chk1-derived peptide (compared to the positive control spot) in the test spot indicates that the test compound suppresses/inhibits an SSB DDR activity. In embodiments, the positive indicator of SSB DDR activity is a phosphorylatable Chk1-derived peptide having SEQ ID NO: 4. In embodiments, the negative indicator of SSB DDR activity is a non-phosphorylatable Chk1-derived peptide having SEQ ID NO: 5. Skilled artisans will recognize that other compounds (such as, but not limited to, other phosphorylatable and non-phosphorylatable peptides) can be used as positive and negative indicators of SSB DDR activity and are intended to be within the scope of the present disclosure.

An embodiment of a high-throughput system for identification of small-molecule modulators of DDR for SSB repair is illustrated in FIG. 5. In the illustrated embodiment, in addition to the negative control spot and the positive control spot, there is an additional suppressor control spot where the spot includes the same phosphorylatable Chk1-derived peptide present in the test spots and the positive control spots (e.g., SEQ ID NO. 4), but the suppressor control spot also includes an inhibitor of a SSB DDR activity, such as compound VE-822 which is an inhibitor of ATR. ATR activity can be detected by measuring incorporate of radiolabeled ATP (e.g., γ-³²P) into a Chk1-derived peptide (e.g., via a phosphorlmager screen). Thus, if a test spot includes a test compound that is a suppressor of Chk1 phosphorylation, the test spot appears similar to the suppressor control spot (e.g., have a similar level of phosphorylation as determined by incorporation of γ-³²P). Depending on the strength of the suppressor, the suppressor control spot or a test spot where a test compound exhibits suppressor activity, the spot may appear similar to negative control spot or it may exhibit a lower level of phosphorylation than the positive control spot but a higher level of phosphorylation signal than the negative control spot. Additional details regarding the high-throughput system for identification of small-molecule modulators of DDR for SSB repair are described in Example 2, below.

If test compounds are identified as modulators using the methods and systems of the present disclosure, the modulatory effect of the compounds can be validated via additional testing, such as, but not limited to using immunoblotting and/or gel electrophoresis or other methodologies to detect, e.g., APE2's 3′-5′ exonuclease activity in vitro; the binding of APE2 Zf-GRF to ssDNA; DNA end resection of FAM-dsDNA-SSB in the HSS system; and the defined SSB-induced ATR-Chk1 DDR pathway activation in the HSS system. Such methods are described in greater detail in the examples below.

A Small Molecule Inhibitor of SSB Signaling and Methods of Using the Inhibitor

The above-described methods and systems of the present disclosure helped identify APE2 inhibitory activity of a known small molecule. Celastrol (chemical name: 3-Hydroxy-9β, 13α-dimethyl-2-oxo-24,25,26-trinoroleana-1(10),3,5,7-tetraen-29-oic acid) is a quinone methide triterpene from Tripterygium wilfordii (also known as Thunder of God Vine). Although has been used as a natural medicine in China for many years (Yang et al., 2006), and evidence suggests that Celastrol exhibits anti-tumor activities in a variety of different types of cancers, it was not known to have any role in SSB signaling and/or the SSB DDR repair pathway. The studies presented in Example 3 below demonstrate that Celastrol surprisingly inhibited the binding of APE2 Zf-GRF to ssDNA in vitro, and the addition of Celastrol to the SSB signaling and repair system described above impaired the defined SSB-induced Chk1 phosphorylation. These data demonstrate that Celastrol has a distinct role in preventing the binding of APE2 Zf-GRF to ssDNA and APE2's critical function in SSB signaling in the HSS system. Additional details are provided in Example 3, below. Based on these results, Celastrol was identified as an inhibitor of SSB DDR activity. Thus, embodiments of the present disclosure also include a method of inhibiting single-strand break (SSB) signaling by contacting a composition of DNA molecules, wherein at least a portion of the DNA molecules have single-strand breaks, with an effective amount of a small molecule inhibitor 3-Hydroxy-9β, 13α-dimethyl-2-oxo-24,25,26-trinoroleana-1(10),3,5,7-tetraen-29-oic acid (Celastrol), where the amount of Celastrol is sufficient to inhibit SSB repair in the DNA molecule.

In embodiments, methods of the present disclosure also include actively inhibiting SSB repair in at least one cell by contacting the at least one cell with an effective amount of 3-Hydroxy-9β, 13α-dimethyl-2-oxo-24,25,26-trinoroleana-1(10),3,5,7-tetraen-29-oic acid. In embodiments the cell is a mammalian cell, such as, but not limited to a human cell. In embodiments, the cell is isolated from a mammal before the contacting step. In embodiments, contacting the cell with the Celastrol includes administration to the mammal. In embodiments the mammal has been diagnosed with a need for inhibiting SSB repair activity prior to the administration to the mammal. In some embodiments the mammal has been diagnosed with a need for treatment of a disorder (such as, but not limited to cancer) related to an SSB repair activity dysfunction prior to the administering step. In embodiments, the method also includes a step of identifying a mammal with a need for inhibiting SSB repair activity.

Additional details regarding the methods and compositions of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following claims.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value, as defined above. In addition, the phrase “about ‘x’ to ‘y’” includes “about ‘x’ to about ‘y’”.

### EXAMPLES

### Example 1

### Use of Cell-Free SSB/HSS System to Demonstrate APE2 Promotion of DDR Pathway in Response to SSB

The present example describes systems and methods used to further study and elucidate the mechanisms of SSB signaling and repair. As the most common type of DNA damage, DNA SSBs are primarily repaired by the SSB repair mechanism. If not repaired properly or promptly, unrepaired SSBs lead to genome stability and have been implicated in cancer and neurodegenerative diseases. However, it remains unknown how unrepaired SSBs are recognized by DNA damage response (DDR) pathway, largely because of the lack a feasible experimental system. Here, we demonstrate that an ATR-dependent checkpoint signaling is activated by a defined plasmid-based site-specific SSB structure in Xenopus HSS (high-speed supernatant) system (FIGS. 1 and 2). Notably, the distinct SSB signaling involves APE2 and canonical checkpoint proteins, including ATR, ATRIP, TopBP1, Rad9, and Claspin, as illustrated in FIG. 4. APE2 interacts with PCNA via its PIP box and preferentially interacts with ssDNA via its C-terminus Zf-GRF domain, a conserved motif found in more than 100 proteins involved in DNA/RNA metabolism such as NEI L3 and Topoisomerase 3. The present example also identifies a novel mode of APE2-PCNA interaction via APE2 Zf-GRF and PCNA C-terminus. M2echanistically, the APE2 Zf-GRF-PCNA interaction facilitates 3′-5′ SSB end resection, checkpoint protein complex assembly, and SSB-induced DDR pathway. The results presented below demonstrate that that APE2 promotes ATR-Chk1 DDR pathway from a single-strand break.

### Materials and Methods

### Experimental Procedures Related to Chromatin and Extracts of Xenopus Laevis

The care and use of Xenopus laevis was approved by the Institutional Animal Care and Use Committee (IACUC) of the University of North Carolina at Charlotte. The preparation of Xenopus LSS, HSS, and NPE were described previously (35,36,39, which are hereby incorporated by reference herein). Immunodepletion of target protein in HSS was performed with a similar procedure as immunodepletion in LSS as previously described (24,32, which are hereby incorporated by reference herein). DNA synthesis analysis was performed as previously described (11,35, which are hereby incorporated by reference herein). ImageQuat software was used to quantify the DNA synthesis from HSS/NPE system. Chromatin fractions were isolated using similar procedures described previously (24,40, hereby incorporated by reference herein). DNA-bound fractions from the HSS system were isolated after spinning extracts through a sucrose cushion (0.9 M sucrose, 2.5 mM MgCl₂, 50 mM KCl, 10 mM HEPES, pH7.7) at 10,000 rpm for 2 minutes at 4° C. with a swinging bucket.

### Preparation of SSB and DSB Plasmid as Well as FAM-SSB Structure

Plasmid pUC19 (SEQ ID NO: 1, +strand) was used as a template for designing a site-specific single-strand break (SSB) structure. There are four recognition sites on pUC19 for Nt.BstNBI, designated as sitel (nt 427-431 on (+) strand), site2 (nt 1177-1181 on (+) strand), site3 (nt 706-710 on (−) strand), and site4 (nt 1694-1698 on (−) strand). The plasmid pS (SEQ ID NO: 2) was generated by mutanting pUC19 on three sites (e.g., site2, site3, and site4) sequentially with three pairs of primers (SEQ ID NOs: 9-14, forward and reverse primers for mutant sites 4, 3, and 2, as indicated in Table 2, below) using QuikChange II XL site-directed mutagenesis kit. The mutations were verified and confirmed by DNA sequencing. Qiagen plasmid midi kit was utilized to obtain large amounts of the pS plasmid.

To generate a defined SSB between C435 and T436, the pS was treated with Nt. BstNBI (10U/μg) for 2 hours at 55° C. and CIP (calf intestine phosphatase, 10U/μg) for 1 hour at 30° C. to remove the 5′-P of T436. The SSB plasmid was purified from agarose via QlAquick gel extraction kit and optionally purified by Phenol-Chloroform extraction. To generate DSB plasmid, the pS was treated with Sbfl-HF at 37° C. and CIP at 37° C. sequentially. The DSB plasmid was purified from agarose via QlAquick gel extraction kit and then optionally purified by Phenol-Chloroform extraction.

For better visualization on gel analysis, a FAM-SSB structure was generated by PCR using the pS as template following by nicking enzyme treatment. The primers having SEQ ID NOs: 15 and 16 were used for PCR amplification. The 70bp dsDNA PCR product (i.e., bp 406-475 of the pS plasmid (SEQ ID NO: 2)) was purified from agarose via QlAquick gel extraction kit and further treated with Nt. BstNBI (10U/μg) for 2 hours at 55° C. and CIP (10U/μg) for 1 hour at 30° C. The FAM-SSB structure was purified from agarose via QlAquick gel extraction kit and then purified by Phenol-Chloroform extraction.

### Recombinant DNA and Pproteins

Recombinant pGEX-4T1-WT APE2-ZF was generated by cloning the ZF domain (nt 1478-1666) of xlAPE2 (GenBank: BC077433, Xenopus Gene Collection IMAGE ID: 4030411), which corresponds to the aa 456-517, into EcoRI- and XhoI-digested pGEX-4T1. Recombinant pET28a-PCNA was generated by subcloning full-length coding region (nt 39-824) of xIPCNA (GenBank: BC057758, Xenopus Gene Collection IMAGE ID:5049027) into BamHI- and NotI-digested pET28a using primer pair F-PCNA and R-PCNA (SEQ ID NOs: 17 and 18, respectively). Recombinant pGEX-4T1-XRCC1 was generated by cloning the coding region (nt 164-2119) of xIXRCC1 (GenBank: BC045032, Xenopus Gene Collection IMAGE ID:5543195) into EcoRI- and XhoI-digested pGEX-4T1 using primers F-XRCC1 and R-XRCC1 (SEQ ID NOs: 19 and 20, respectively). Recombinant pGEX-4T1-APE1 was generated by cloning the coding region (nt 119-1069) of xlAPE1 (GenBank: BC072056, Xenopus Gene Collection IMAGE ID: 4202632) into BamHI- and XhoI-digested pGEX-4T1.

Point mutants of recombinant DNA were generated with QuikChange IIXL Site-Directed Mutagenesis kit (Agilent). Recombinant plasmids were made via QlAprep spin miniprep kit following vendor's standard protocol. Myc-tagged recombinant proteins were generated with various pCS2+MT-derived recombinant plasmids and TNT SP6 Quick Coupled Transcription/Translation System (Promega) according to the manufacturer's protocol. GST-or His-tagged recombinant proteins were expressed and purified in E. coli DE3/BL21 following vendor's standard protocol. Purified recombinant proteins were confirmed on coomassie-stained SDS-PAGE gels with a range of BSA standards and a pre-stained protein ladder.

### Immunoblotting Analysis and Antibodies

Anti-XRCC1 antibodies were raised in rabbits against GST-XRCC1 (Cocalico Biologicals). Anti-Xenopus APE2 antibodies was described previously (24). Antibodies against ATR and Claspin were provided by Dr. Karlene Cimprich (33,41). Antibodies against ATRIP, Rad9, and Rad17 were provided by Dr. Howard Lindsay (42). Antibodies against TopBP1 and RPA32 were provided by Dr. Matthew Michael (11). Antibodies against PARP1 was provided from Dr. Yoshiaki Azuma (43). Antibodies against human APE2 was provide by Drs. Yusaku Nakabeppu and Daisuke Tsuchimoto (44). Antibodies against RPA32 phosphorylation at Ser33 and Rad17 phosphorylation at Ser645 were purchased from Bethyl Laboratories. Antibodies against Chk1 phosphorylation at Ser345 were purchased from Cell Signaling Technology. Antibodies against Histone 3 were purchased from Abcam. Antibodies against Chk1, GST, His, Myc, PCNA, and Tubulin were purchased from Santa Cruz Biotechnology. Antibodies against human Chk1 and human RPA32 were purchased from Cell Signaling Technology and Thermo Scientific, respectively.

### GST Pulldown Assays

For the GST-pull-down experiments from HSS, 5 μg of GST or GST-tagged recombinant proteins were added to 200 μL Xenopus HSS. After an hour of incubation, an aliquot of egg extract mixture was collected as Input and the remaining extract mixture was diluted with 200 μL Interaction Buffer (100 mM NaCl, 5 mM MgCl2, 10% (vol/vol) glycerol, 0.1% Nonidet P-40, 20 mM Tris-HCl at pH 8.0). Then, 30 μL of glutathione beads that were resuspended in 200 μL interaction buffer were added to the diluted egg extracts. After 1 h incubation at room temperature, beads were washed with Interaction Buffer two times. Then, the bead-bound fractions and Input were analyzed via immunoblotting.

For the GST-pull-down experiment from a buffer, 5 μg of GST or GST-tagged recombinant proteins, and 5 μg of WT or mutant His-tagged PCNA were added to 200 μL Interaction Buffer. After an hour of incubation, an aliquot of the mixture was collected as Input and the remaining mixture was supplemented with 100 μL interaction buffer that contains 30 μL glutathione beads. After 1h incubation at room temperature, beads were washed with Interaction Buffer two times. Then, the bead-bound fractions and the Input were analyzed via immunoblotting.

Analysis of DNA Repair Products from the HSS System

Nuclease-free water was added to each reaction of HSS that was incubated with SSB or DSB plasmid to a total of 300 μL. Equal volume of phenol-chloroform was added to the mixture and resuspended up and down 5 times and spin for 5 minutes at 13,000 rpm at room temperature. The top aqueous layer was transferred to a new tube and repeat phenol-chloroform extraction. Then sodium acetate (pH5.0, a final concentration of 0.3 M) and glycogen (a final concentration of 1 μg/pl) as well as ethanol (100%, 2.5-fold volume) were added to the aqueous solution, which was incubated for 30 minutes at −70° C. The mixture was centrifuged for 15 minutes at 13,000 rpm at room temperature. The pellet was washed by cold 70% ethanol and air-dry for 30 minutes before resuspension with nuclease-free water. Then the purified DNA repair products were analyzed on agarose gel and stained with ethidium bromide.

### SSB End Resection Assays in the HSS System

The FAM-SSB structure was added to mock- or APE2-depleted HSS, which was supplemented with WT/mutant Myc-tagged APE2, respectively. After different time of incubation at room temperature, reactions were quenched with equal volume of TBE-Urea Sample Buffer (Invitrogen), denatured at 95° C. for 5 minutes, and cooled at 4° C. for 2 minutes. Samples were examined on 20% TBE-Urea gel and imaged on Typhoon 8600 and viewed using ImageQuant software.

### In Vitro Exonuclease Assays

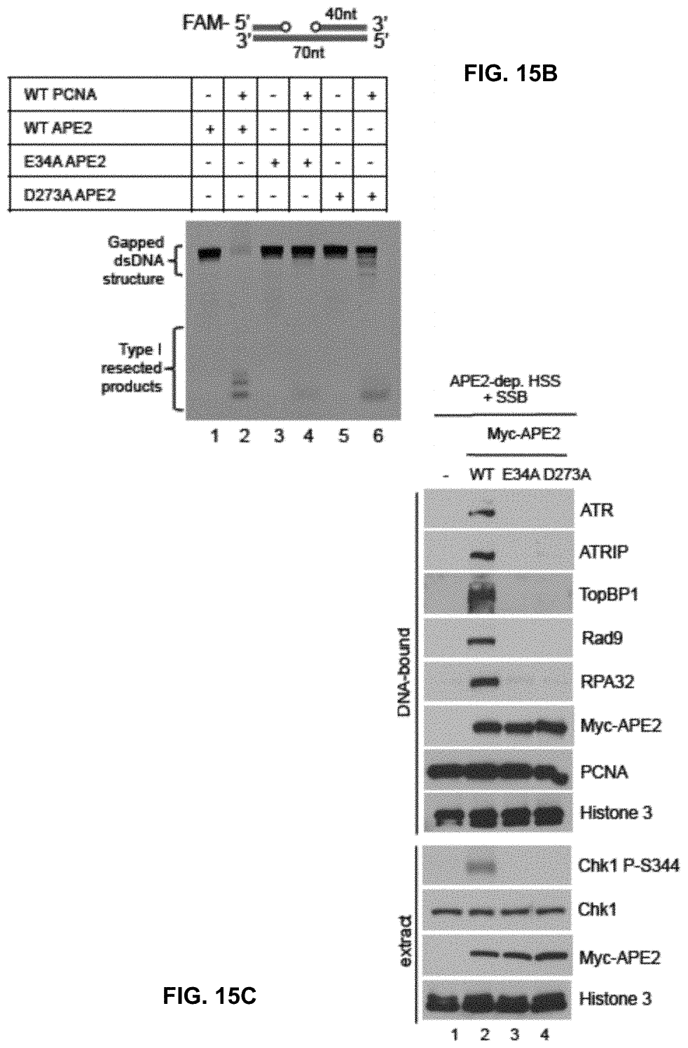

Previous biochemistry analysis has indicated that APE1 can resect nicked dsDNA into 1-3 nt gap in the 3′-5′ direction in vitro (45). To generate a FAM-labeled gapped structure, it was found that the recombinant GST-APE1 resected FAM-labeled SSB substrate in the 3′-5′ direction in a dose-dependent manner (see FIG.15A and related discussion below). Thus, this APE1-pretreated FAM-labeled gapped substrate was utilized for APE2 exonuclease analysis in vitro. For the in vitro exonuclease assays, the FAM-SSB substrate was pretreated with recombinant APE1 in exonuclease buffer (20 mM KCl, 10 mM MCl₂, 2 mM DTT, 50 mM HEPES, pH 7.5) at 95° C. for 20 minutes, followed by phenol-chloroform extraction and purification. This APE1-treatment method is derived and modified from a method described previously (45, which is hereby incorporated by reference herein). The purified gapped dsDNA structure (50 nM) was incubated in 1× reaction buffer (50 mM NaCl, 1 mM TCEP, 1 mM MnCl₂, 10 mM Tris-HCl, pH 8.0) with different combinations of purified recombinant proteins. After a 30-minute incubation at 37° C., reactions were quenched with equal volume of TBE-Urea Sample Buffer, denatured at 95° C. for 5 minutes, and cooled at 4° C. for 2 minutes. Samples were loaded and resolved on a 20% TBE-Urea gel. Gels were imaged using a Typhoon imager (GE Healthcare) and viewed using ImageJ.

### DNA Binding Assays

For the ssDNA binding assays in a buffer using GST or GST-tagged proteins, 40 μL of biotin-ssDNA (SEQ ID NO: 21, 80 nt, 100 μM) was added to 40 μL of Streptavidin Dynabeads in 2x B&W Buffer (2M NaCl, 1 mM EDTA, 10 mM Tris-HCl, pH7.5), and incubated for 15 minutes at room temperature. After separating beads from buffer, the beads were washed by 2x B&W Buffer for three times and resuspended in 100 μL of Buffer B (80 mM NaCl, 20 mM p-Glycerophosphate, 2.5 mM EGTA, 0.01% NP-40, 10 mM MgCl₂, 100ug/mL BSA, 10 mM DTT, and 10 mM HEPES-KOH, pH7.5). Then 20 μg of GST or GST-tagged proteins in 100 μL of Buffer B was added to the 80 nt-ssDNA-coupled beads in 100 μL of Buffer B. After a 30-minute incubation at 4° C., an aliquot of mixture was collected as input, and the beads were washed for three times with Buffer A (80 mM NaCl, 20 mM p-Glycerophosphate, 2.5 mM EGTA, 0.01% NP-40, and 10 mM HEPES-KOH, pH7.5). The Input and Bead-bound fractions were examined via immunoblotting analysis.

For the ssDNA binding assays in the HSS system, different lengths of Biotin-ssDNA (i.e., 0, 10 (SEQ ID NO: 25), 20 (SEQ ID NO: 24), 40 (SEQ ID NO: 23), 60 (SEQ ID NO: 22), and 80 (SEQ ID NO: 21)) were coupled to Streptavidin Dynabeads using a similar approach as above described, and resuspended in 200 μL of Buffer B. Then, the 200 μL of beads coupled with ssDNA was added to equal volume of diluted HSS (1:1 v/v dilution with Buffer B). After 1-hour incubation, an aliquot of the mixture was collected as Input, and the beads were washed with Buffer A three times. The Input and Bead-bound fractions were examined via immunoblotting analysis.

For the gapped DNA binding assays with purified proteins, biotin-gapped DNA structure was prepared in a similar approach to that for the FAM-gapped DNA structure with the exception that biotin was covalently linked to the 5′ side of one primer. The coupling of biotin-gapped DNA structure to Streptavidin Dynabeads, protein incubation and bead washing process were performed following the protocol for ssDNA binding analysis in a buffer as described above.

### Cell Culture, Treatment, and Analysis

Human U2OS cells were purchased from the American Type Culture Collection (ATCC) and cultured in Dulbecco's Modified Eagle Medium (DMEM, Life Technologies) supplemented with 10% fetal bovine serum (FBS, Invitrogen), penicillin (100 U/mL) and streptomycin (100 μg/mL, Life Technologies) in incubator at 37° C. and 5% CO₂. For G1 synchronization, cells were first blocked in S phase with 2 mM thymidine for 24 hours and released for 4 hours, then blocked in M phase with 100ng/mL nocodazole for 12 hours and released for 6 hours, and finally blocked in G1 phase with DM EM without FBS for 96 hours. Cells were treated with VE-822 (5 μM) in culture medium for 1 hour followed by hydrogen peroxide treatment (1.25 mM) for 4 hours. Then cells were collected and split for immunoblotting analysis and FACS analysis. For immunoblotting analysis, equal total proteins of cell lysates (10 μg per lane) were loaded and Tubulin was used for loading control. For FACS analysis, cells were further fixed and stained with DAPI followed by cell cycle profiling using FACS machine (BD LSRFortessa) following manufacturer's standard protocol.

### Quantification

ImageQuant software was utilized to quantify the incorporation of [³²P-α]-dATP to newly synthesized DNA from the HSS/NPE system. ImageQuant was used to visualize gels from SSB end resection assays. ImageJ was utilized to view gels from exonuclease assays.

### Results

A Defined SSB Structure can be Repaired in the HSS System

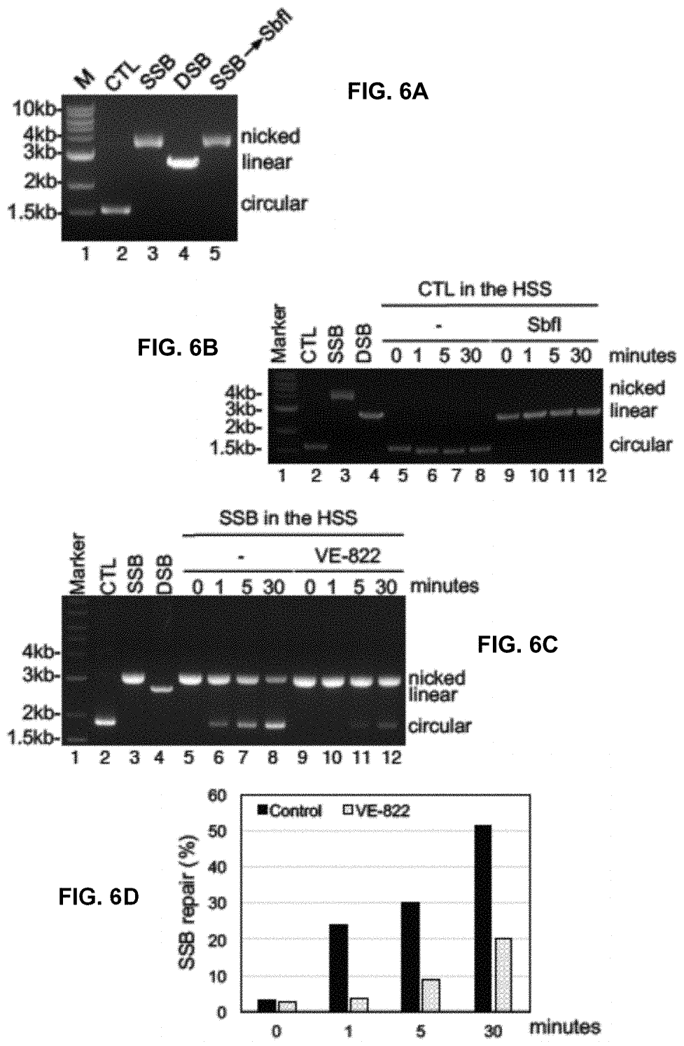

In the present example, a pUC19-derived plasmid, pS, was generated that contains only one recognition sequence for a nicking endonuclease Nt.BstNBI (FIG. 1). pS was treated by Nt.BstNBI and calf intestinal alkaline phosphatase (CIP) sequentially to generate a SSB structure containing only one nick between dC435 and dT436 in the “+”strand with 3′-OH and 5′-OH at both ends. pS was treated with restriction enzyme Sbfl and CIP sequentially to generate a DSB structure (FIG. 1). The SSB, DSB and CTL (control) plasmid structures are distinguished on agarose gel based on their structure-dependent mobility shift (FIG. 1 and FIG. 6A). Because the nick is in the recognition sequence of Sbfl, the SSB structure was resistant to further Sbfl treatment, as expected (FIG. 6A).

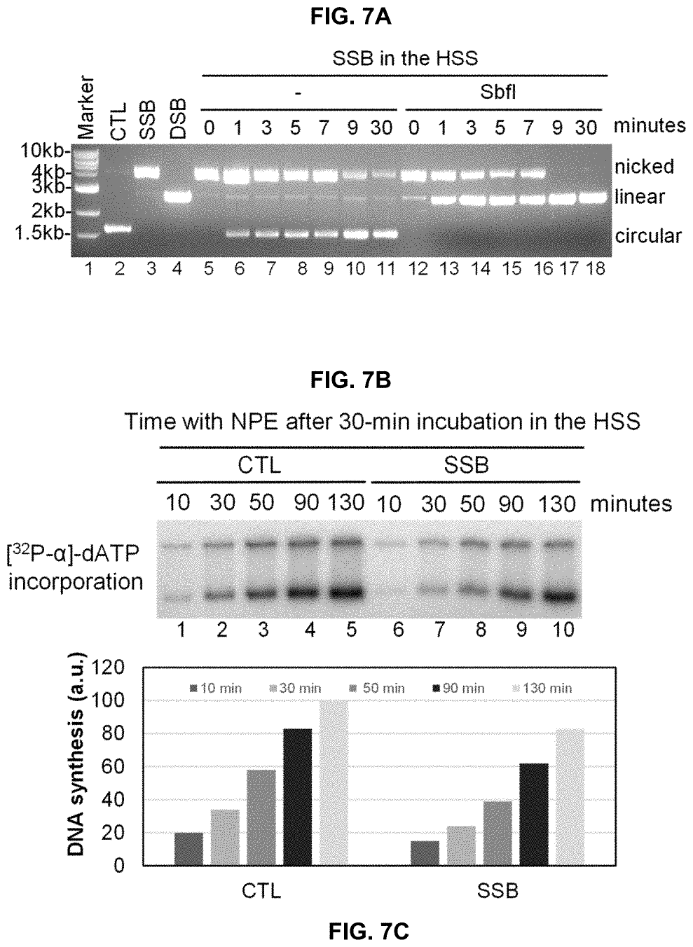

To examine SSB repair process in the HSS system, it was found that the nicked version was converted into a circular version after incubation in HSS for different lengths of time, indicating that the SSB structure is gradually repaired in HSS (Lane 6-11, FIG. 7A). Moreover, the circular repair products of SSB structure from HSS were catalyzed by Sbfl into a linear version (Lane 12-18, FIG. 7A). Strikingly, a portion of the nicked version of SSB structure developed Sbfl sensitivity at beginning time points (1, 3, 5 and 7 minutes), whereas all SSB structures were sensitive to Sbfl at late time points (9 and 30 minutes) (FIG. 7A). These observations suggest that, after HSS incubation, the defined SSB structure is either repaired into a circular version or converted into an intermediate product with a normal Sbfl recognition sequence but with a nick and/or gap at another location. The CTL plasmid isolated from the HSS was catalyzed by Sbfl into linear versions at all time points (FIG. 6B). Circular plasmid DNA can be assembled into pre-Replication Complex in the HSS, but cannot continue its DNA synthesis without addition of the NPE supplying S-phase CDKs (37). To confirm the defined SSB structure is indeed repaired in the HSS, they were examined using the Xenopus HSS/NPE system (37). Similar to CTL plasmid, the defined SSB structure can be replicated efficiently in the HSS/NPE system (only ˜17% reduction), suggesting that most SSB structures have been repaired after a 30-min HSS incubation (FIG. 7B-7C).

A distinct ATR-Chkl DNA damage response pathway is induced by a defined SSB structure in the HSS system

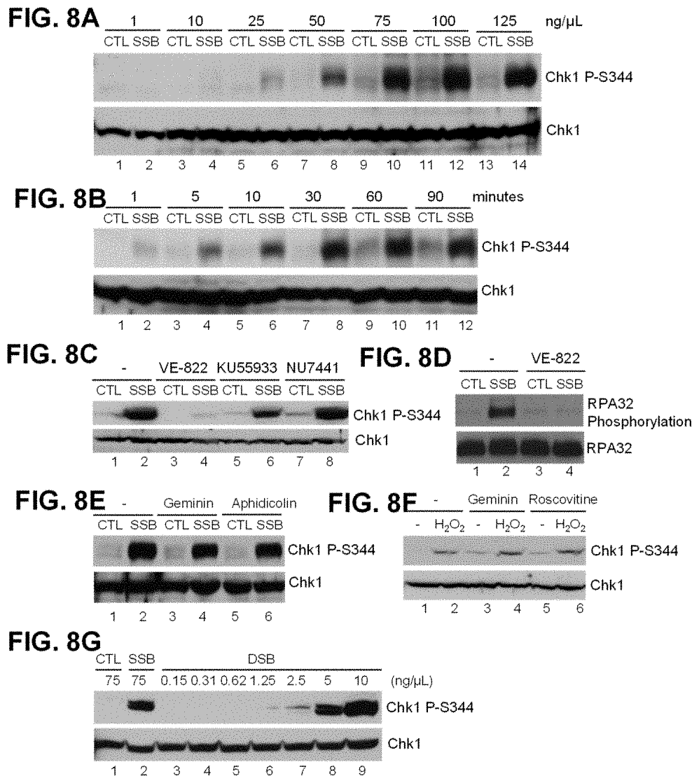

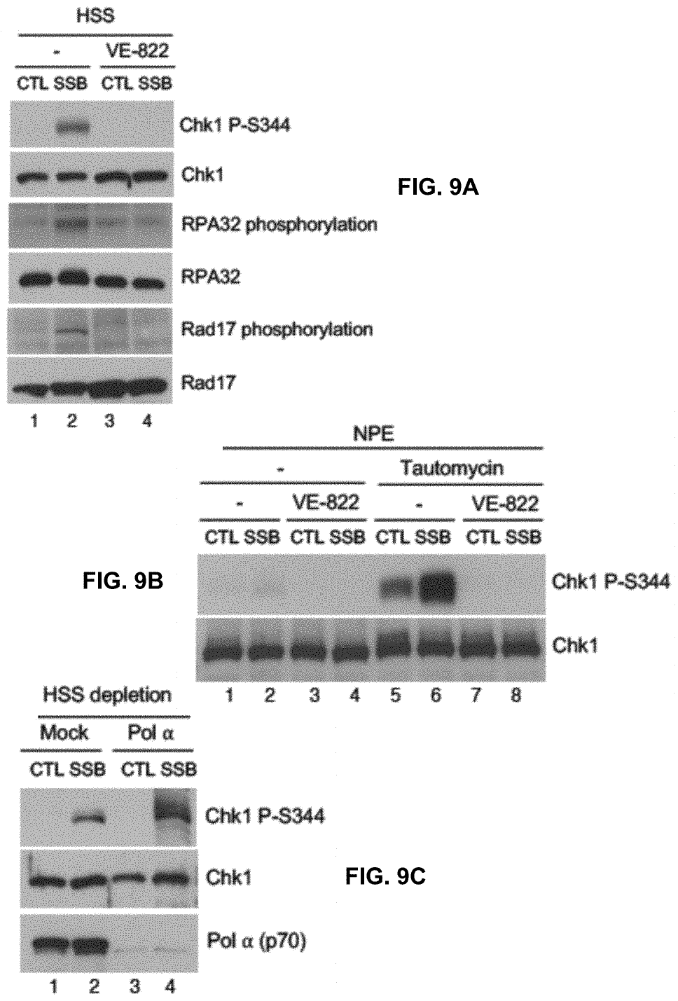

To determine whether a defined SSB structure triggers a DDR pathway, different concentrations of SSB or CTL plasmid structure were added to HSS, followed by immunoblotting analysis after a 30-minute incubation. This revealed that 75ng/μL (43 nM) of the defined SSB plasmid, but not CTL plasmid, triggered a robust Chk1 phosphorylation at S344 in the HSS (equivalent to Chk1 phosphorylation at S345 in humans), suggesting that the SSB structure induces a unique checkpoint signaling in the HSS in a dose-dependent manner (FIG. 8A). A time-dependence analysis found that the SSB-induced Chk1 phosphorylation appeared at 5 minutes, and peaked at 30 minutes after incubation in the HSS (FIG. 8B). Whereas ATM specific inhibitor KU55933 and DNA-PK specific inhibitor NU7441 almost had no effect on SSB-induced Chk1 phosphorylation, the addition of ATR specific inhibitor VE-822 compromised the SSB-induced Chk1 phosphorylation, suggesting that the SSB signaling is ATR-dependent (FIG. 8C). Consistent with this observation, RPA32 phosphorylation and Rad17 phosphorylation were induced by the defined SSB in the HSS in a VE-822 sensitive manner FIG. 8D and FIG. 9A). In addition, it was verified that potential DSB contamination in the SSB plasmid is less than ˜1% by quantification. The defined DSB structure required at least 5 ng/μL (3 nM) to trigger a robust Chk1 phosphorylation in the HSS, suggesting that the defined SSB-induced Chk1 phosphorylation is not due to confounding DSB in the SSB preparation (FIG. 8G).

Although DNA replication is required for ATR-Chk1 checkpoint activation in response to stalled replication forks and oxidative stress (32,33,46), the defined SSB-induced Chk1 phosphorylation was not impaired by the addition of MCM helicase inhibitor recombinant geminin protein, suggesting that DNA replication is dispensable for the SSB-induced Chk1 phosphorylation in the HSS system (FIG. 8E). This observation is consistent with the deficiency of plasmid DNA replication elongation in Xenopus HSS without addition of NPE (37). Aphidicolin, a small molecule inhibitor of DNA polymerase alpha, delta, and epsilon, can induce stalled DNA replication forks to trigger ATR activation in the Xenopus LSS system (11,47,48). However, the present data show that the SSB-induced Chk1 phosphorylation was not affected by the addition of aphidicolin (FIG. 8E). Furthermore, the SSB-induced Chk1 phosphorylation was not affected when Pol alpha was depleted in the HSS system (FIG. 9C). This feature of the SSB-induced replication-independent Chk1 phosphorylation in the HSS system is similar to the DSB-mimicking structure AT70-induced Chk1 phosphorylation in a replication-independent manner in the LSS system (34,49). Moreover, Chk1 phosphorylation was triggered by hydrogen peroxide when HSS was supplemented with sperm chromatin; however, the addition of geminin and CDK inhibitor rescovitine had no effect on the hydrogen peroxide-induced Chk1 phosphorylation in the HSS (FIG. 8F).

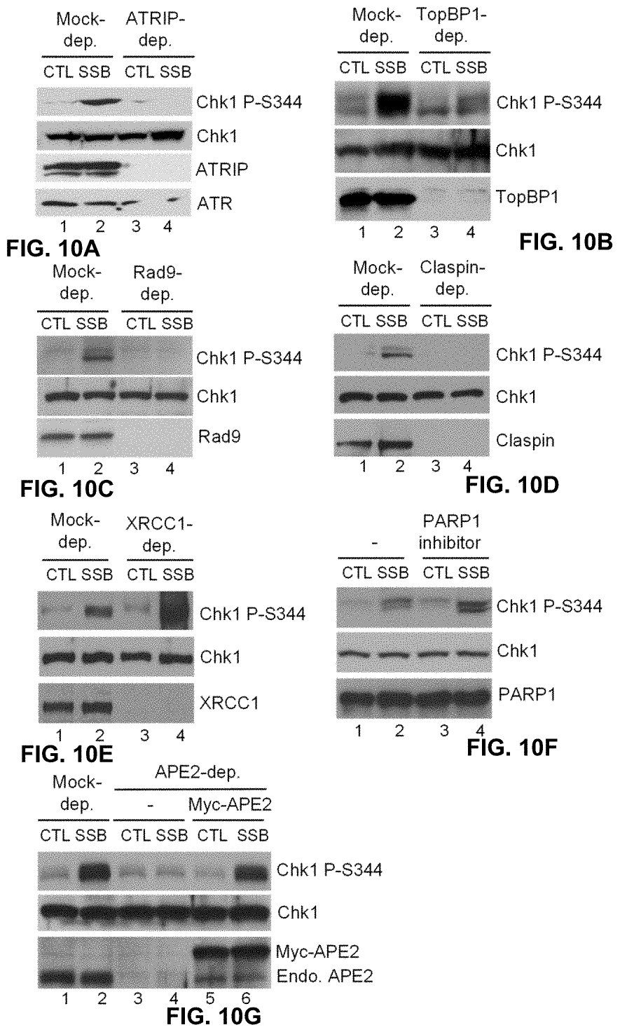

To further determine whether the defined SSB-induced Chk1 phosphorylation is ATR-dependent, endogenous ATRIP was removed via immunodepletion using anti-ATRIP antibodies which also co-depleted endogenous ATR, as ATR and ATRIP form a tight complex in Xenopus egg extracts (FIG. 10A) (24). Notably, the SSB-induced Chk1 phosphorylation was compromised when ATRIP and most of ATR were absent in the HSS system (FIG. 10A). This observation strongly suggests that the defined SSB structure triggers ATR activation. Together, the above observations suggest that a defined site-specific SSB structure triggers ATR-Chk1 DDR pathway activation in a dose- and time-dependent, but replication-independent, fashion in the Xenopus HSS system.

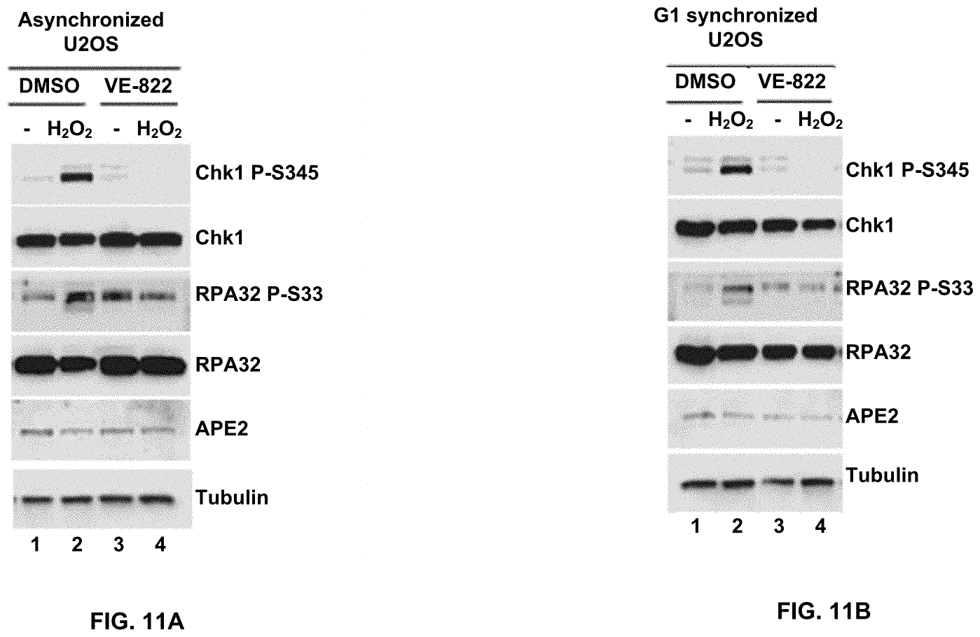

To identify the significance of SSB-induced ATR activation, whether SSB repair is affected by ATR in the HSS system was examined. It was found that the addition of ATR specific inhibitor VE-822 compromised the SSB repair in the HSS system (FIG. 6C-6D). This observation strongly suggests that the SSB-induced ATR activation is important for SSB repair. To address the physiological relevance of the SSB-induced ATR DDR pathway, whether hydrogen peroxide induces ATR-Chk1 DDR in mammalian cells was tested. A recent study using human U2OS cells demonstrated that ATR kinase is activated in G1 phase to facilitate the repair of ionizing radiation-induced DNA damage (50). Notably, the present data showed that hydrogen peroxide triggers Chk1 phosphorylation and RPA32 phosphorylation in asynchronized U2OS cells (Lane 1 and 2, FIG.11A). Importantly, addition of ATR specific inhibitor VE-822 prevented the hydrogen peroxide-induced Chk1 phosphorylation and RPA32 phosphorylation in asynchronized U2OS cells (Lane 3 and 4, FIG. 11A). It was also observed that hydrogen peroxide induced ATR-Chk1 DDR in G1 synchronized U2OS cells (FIG. 11B). These observations in human U2OS cells suggest that the observations in Xenopus egg extracts system are very likely conserved in humans, demonstrating the physiological relevance of the findings obtained using Xenopus HSS system.

### APE2 is Involved in the Defined SSB-Induced DDR Pathway Activation

Previous studies have shown that TopBP1, Rad9, and Claspin are canonical checkpoint proteins required for the ATR-Chk1 DDR pathway (8,34,40). Notably, the defined SSB-induced Chk1 phosphorylation was compromised when TopBP1, Rad9 or Claspin was immunodepleted in the HSS, respectively, suggesting the requirement of these checkpoint proteins for SSB signaling (FIGS. 10B-10D). XRCC1 and PARP1 have been demonstrated as associated with SSB repair (43,51,52). The present study found that the defined SSB-induced Chk1 phosphorylation was enhanced when XRCC1 was immunodepleted in HSS or when PARP1 specific inhibitor (4-Amino-1,8-naphthalimide) was added to HSS, respectively (FIGS. 10E and 10F). An interpretation of these observations is that the SSB signaling is enhanced when SSB repair is impaired by XRCC1 depletion or the addition of PARP1 inhibitor. Notably, the defined SSB-induced Chk1 phosphorylation is compromised in APE2-depleted HSS (FIG. 10G). Importantly, recombinant WT Myc-tagged APE2 protein rescued the deficiency of Chk1 phosphorylation in APE2-depleted HSS (FIG. 10G). These observations suggest that APE2 be required for the defined SSB-induced Chk1 phosphorylation in the HSS system.

APE2 Zf-GRF Associates with PCNA C-Terminal Motif as a Distinct Mode of APE2-PCNA Interaction

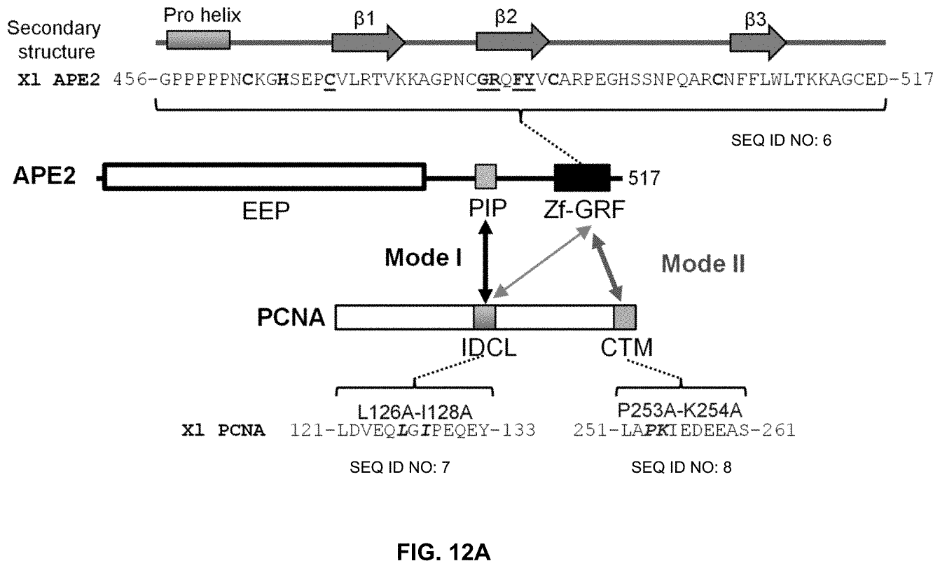

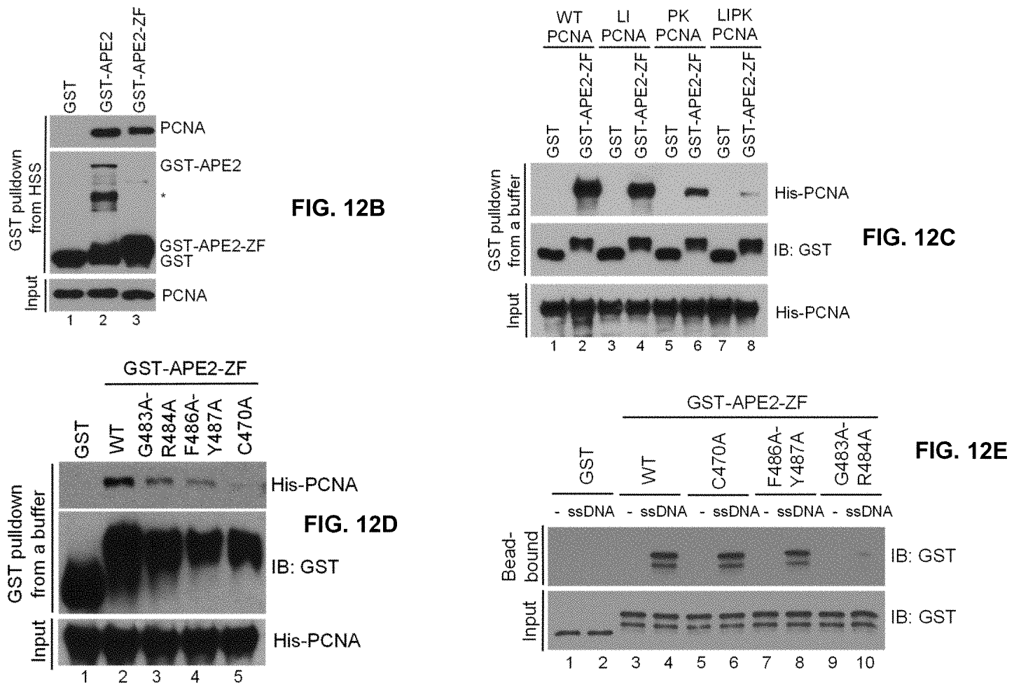

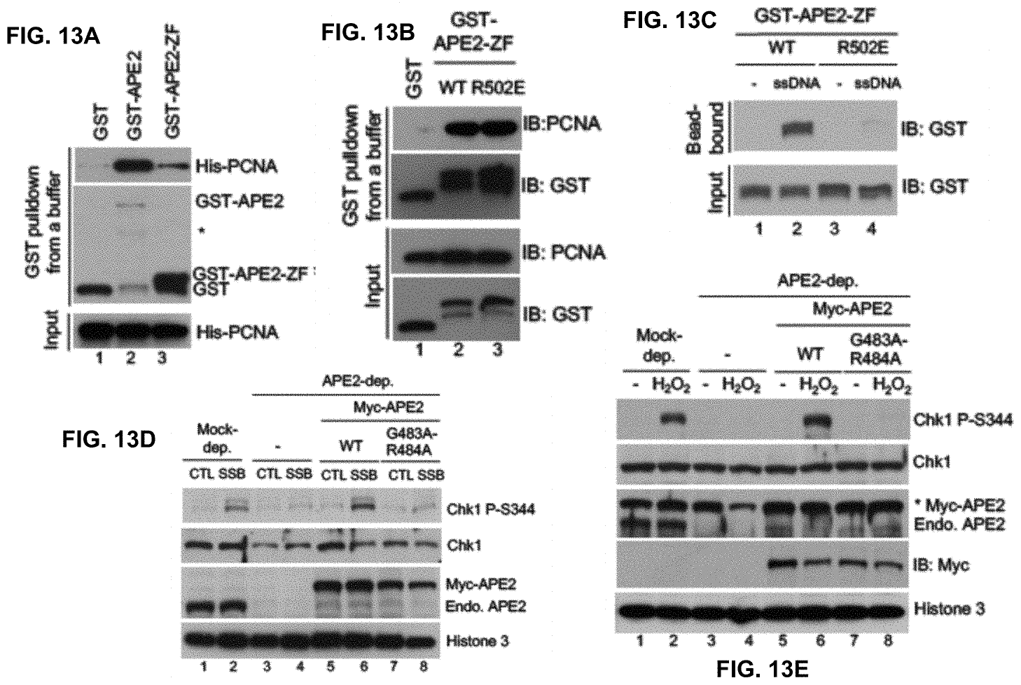

APE2 interacts with PCNA's IDOL motif via its PIP box in yeast, Xenopus, and humans (FIG. 12A) (24,30,31). Consistent with previous studies, GST-pulldown assays in the present studies demonstrated that PCNA associated with GST-APE2, but not GST, from Xenopus HSS, suggesting that APE2 associates with PCNA in the HSS (FIGS. 12A and 12B). Surprisingly, PCNA was also pulled down in the HSS by GST-APE2-ZF, which does not contain the PIP box, suggesting that APE2 Zf-GRF associates with PCNA in a PIP box-independent manner (FIG. 12B). To further confirm the interaction between APE2 Zf-GRF with PCNA, GST-pulldown assays were done with recombinant PCNA protein and found that both GST-APE2 and GST-APE2-ZF, but not GST, pulldown recombinant PCNA in an interaction buffer, suggesting a possible direct interaction between APE2 Zf-GRF and PCNA (FIG. 13A). Notably, interacting with APE2 Zf-GRF was compromised in PK PCNA (P253A-K254A PCNA) and almost completely prevented in LIPK PCNA (L126A-1128A-P253A-K254A PCNA), suggesting that the PCNA C-terminal motif (CTM) plays an important role in Zf-GRF association (FIG. 12A and 12C).

To identify critical residues within APE2 Zf-GRF for its interaction with PCNA, three point mutants were generated in GST-APE2-ZF (i.e., G483A-R484A, F486A-Y487A, and C470A). GST-pulldown assays demonstrated that G483A-R484A, F486A-Y487A, and C470A GST-APE2-ZF failed to efficiently interact with recombinant PCNA in an interaction buffer, in comparison to WT GST-APE2-ZF (FIG. 12D). Because APE2 Zf-GRF associates with ssDNA (32), it was intended to distinguish its interaction with PCNA from its association with ssDNA. 80 nt ssDNA tagged with Biotin in the 5′ side were coupled to streptavidin beads, and it was found that WT GST-APE2-ZF, but not GST, interacts with ssDNA (FIG. 12E). Importantly, G483A-R484A APE2-Zf-GRF is deficient in ssDNA interaction, whereas F486A-Y487A and C470A APE2-Zf-GRF are proficient for ssDNA binding (FIG. 12E). In addition, R502E APE2 Zf-GRF is proficient in PCNA interaction although R502E APE2 Zf-GRF is deficient for ssDNA interaction and its exonuclease activity (32), (FIGS. 13B and 13C). These observations suggest that APE2 Zf-GRF interaction with PCNA CTM is distinguished from its interaction with ssDNA. As the interaction of APE2 PIP box with PCNA IDOL motif is the first mode of APE2-PCNA interaction, the APE2 Zf-GRF interaction with PCNA CTM was designated as the second distinct mode of APE2-PCNA interaction (FIG. 12A).

### APE2 Zf-GRF-PCNA CTM Interaction is Instrumental for 3′-5′ SSB End Resection, Assembly of a Checkpoint Protein Complex to SSB Sites, and SSB Signaling

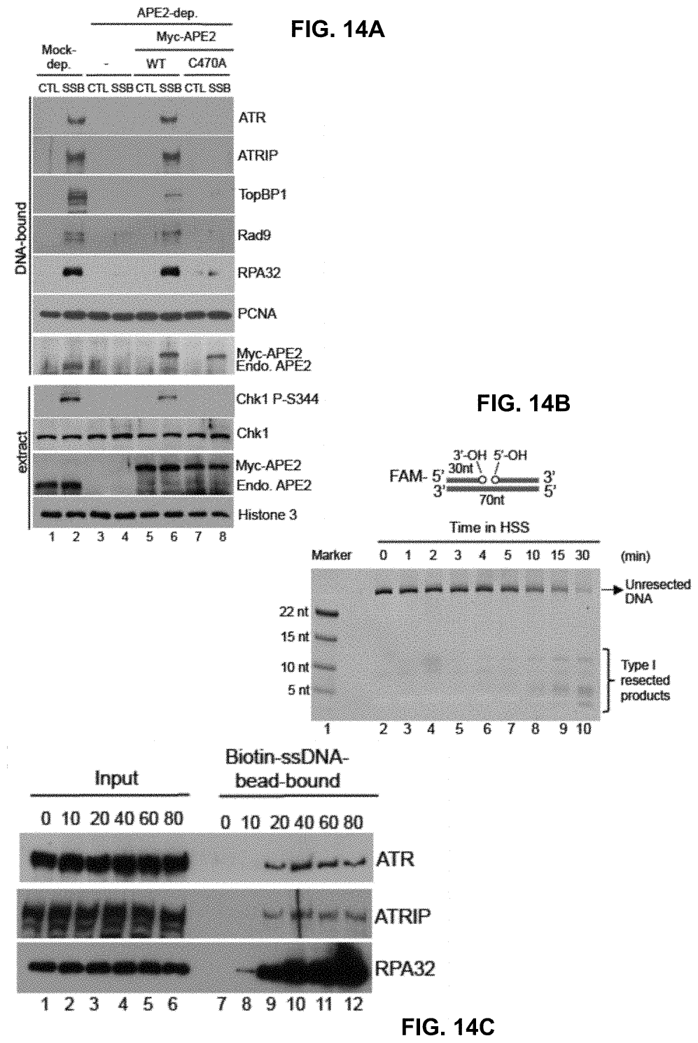

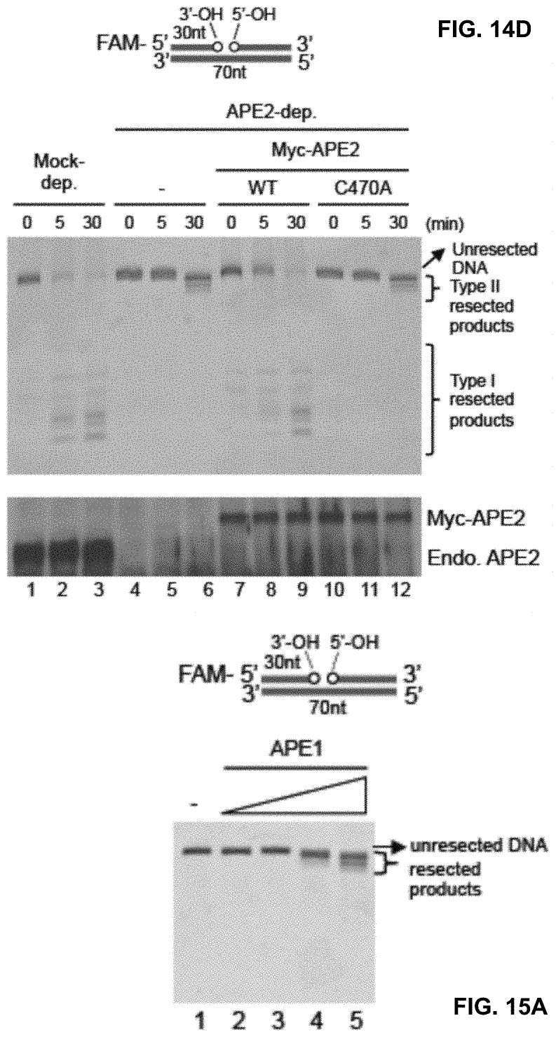

To characterize the biological significance of APE2 Zf-GRF interaction with PCNA CTM motif, WT or C470A Myc-tagged APE2 was added back to APE2-depleted HSS, and WT, but not C470A, APE2 was found to rescue the SSB-induced Chk1 phosphorylation in APE2-depleted HSS (FIG. 14A), suggesting that the APE2 Zf-GRF-PCNA CTM interaction is important for the SSB-induced ATR-Chk1 DDR pathway in the HSS system (FIG. 14A). Moreover, G483A-R484A APE2, which is deficient in interaction with ssDNA and PCNA CTM motif (FIGS. 12D and 12E), also failed to rescue the SSB-induced Chk1 phosphorylation in APE2-depleted HSS (FIG. 13D). Furthermore, WT APE2, but not G483A-R484A APE2, rescued the hydrogen peroxide-induced Chk1 phosphorylation in APE2-depleted LSS system (FIG. 13E). These observations suggest that APE2 Zf-GRF interaction with PCNA CTM is important for the SSB signaling.

Next, the DNA-bound fractions were isolated from HSS and the abundance of checkpoint proteins via immunoblotting analysis were examined. Although PCNA was recruited to both CTL and SSB plasmid, a checkpoint protein complex, including ATR, ATRIP, TopBP1, and Rad9, was assembled onto SSB plasmid, but not CTL plasmid, in the HSS system (DNA-bound fractions, Lane 1-2, FIG. 14A). Notably, APE2 preferentially associated with SSB plasmid, but not CTL plasmid, and RPA32 was also hyperloaded to SSB plasmid, but not CTL plasmid, in the HSS system, suggesting that the SSB plasmid is resected by APE2 into ssDNA for RPA binding and the assembly of the checkpoint protein complex assembly (Lane 1-2, FIG. 14A). When APE2 was removed via immunodepletion, the recruitment of RPA32, ATR, ATRIP, TopBP1, and Rad9 to SSB plasmid was compromised, further supporting the critical role of APE2 in the SSB end resection (Lane 3-4, FIG. 14A). Importantly, WT APE2, but not C470A APE2, rescued the recruitment of RPA32, ATR, ATRIP, TopBP1, and Rad9 to SSB in APE2-depleted HSS (Lane 5-8, FIG. 14A). Of note, similar to WT APE2, C470A APE2 was also recruited to SSB site efficiently in HSS, suggesting that APE2 PIP box interaction with PCNA IDCL motif is sufficient for the recruitment of APE2 to SSB site (FIG. 14A). Together, this evidence demonstrates that the APE2 Zf-GRF interaction with PCNA CTM is important for the checkpoint protein complex assembly onto SSB site and the SSB-induced ATR-Chk1 DDR pathway activation, but is dispensable for APE2 recruitment to SSB sites, in the HSS system.

To further investigate the SSB end resection by APE2 per se, a FAM-labeled dsDNA was generated in which a SSB is localized at a defined location in the upper strand, designated as FAM-SSB (70bp in total, FIG. 14B). After FAM-SSB was incubated in HSS for different time points, samples were examined via urea-denaturing PAGE electrophoresis. The FAM-SSB was resected in the 3′ to 5′ direction into Type I resected products in HSS (FIG. 14B). Because the resected products are arranged from ˜4 nt to ˜12 nt, we estimated that the SSB structure is resected ˜18 nt to 26 nt in the 3′ to 5′ direction in the HSS system. Importantly, the 3′-5′ end resection of FAM-SSB was significantly compromised when APE2 was removed in HSS (top panel, lane 1-6, FIG. 14D). Interestingly, the FAM-SSB was still resected only 1 nt-3 nt, designated as Type II resected products, in APE2-depleted HSS (top panel, lane 6, FIG. 14D). Although WT APE2 and C470A APE2 are added to similar concentrations in APE2-depleted HSS (bottom panel, FIG. 14D), WT APE2 but not C470A APE2 rescued the deficiency of SSB end resection of FAM-SSB in APE2-depleted HSS (top panel, FIG. 14D), suggesting that the APE2 Zf-GRF-PCNA CTM interaction is critical for the 3′-5′ SSB end resection in the HSS system.

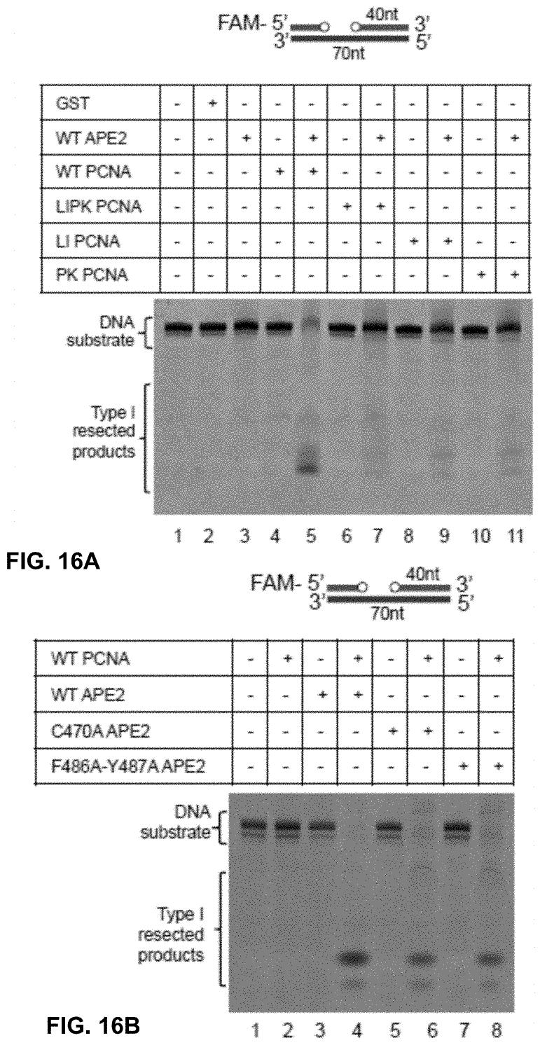

Using reconstitution system with purified proteins in vitro, DNA end resection of a gapped dsDNA structure was examined, in which the FAM-SSB was pretreated with recombinant APE1 to generate approximately 1-3 nt gap (FIG. 15A). The gapped dsDNA was catalyzed into Type I resected products by recombinant APE2 with the presence of WT PCNA, but not LIPK PCNA, LI PCNA, or PK PCNA, suggesting that PCNA IDCL and CTM are all important for APE2's exonuclease activity in vitro (FIG. 16A). Surprisingly, the gapped dsDNA structure was still resected by C470A APE2 or F486A-Y487A APE2 to some extent similar to WT APE2 with the presence of WT PCNA (FIG. 16B).

The different requirement of APE2 Zf-GRF interaction with PCNA CTM for SSB end resection in the HSS and in purified protein system in vitro may be because of a previously unidentified negative regulatory factor in the HSS system. It is believed that the Zf-GRF-PCNA CTM interaction may be needed to overcome such inhibition of SSB end resection in the HSS system. Together, the above data suggest that the APE2 Zf-GRF-PCNA interaction promotes the ATR-Chk1 DDR pathway activation from a site-specific SSB structure in a cell-free eukaryotic system such as illustrated in the schematic shown in FIG. 4.

### Discussion

It is believed that this is the first report that a defined SSB triggers the ATR-Chk1 DDR pathway in a eukaryotic experimental system. Based on evidence in this study, a working model for the SSB-induced ATR-Chk1 DDR pathway is proposed as illustrated in FIG. 4: (a) 3′-5′ SSB end resection is initiated into a small gap (˜1 nt-3 nt) by a mechanism to be determined; (b) APE2 is recruited by PCNA via its PIP box (24), and activated by APE2 Zf-GRF interaction with ssDNA (32) and PCNA C-terminus for SSB end resection continuation (evidence provided here); (c) a longer stretch of ssDNA (˜18-26 nt) is generated and bound with RPA, leading to the assembly of ATR-ATRIP, TopBP1, and 9-1-1 complex to activate DDR; (d) activated ATR phosphorylates its substrates including Chk1 and RPA32; and (e) activated ATR DDR pathway is important for SSB repair.

The SSB signaling system of the present disclosure requires only HSS but not the addition of NPE, which is different from previously established reconstitution systems, such as ATR DDR pathway activation by 3′-primed ssDNA or defined ICLs using Xenopus HSS and NPE combined systems (53,54). The defined SSB structure is resected by APE2 in the 3′ to 5′ direction to generate a longer stretch of ssDNA, which is in line with the previously established general model for ATR-Chk1 DDR pathway activation (7,8,14,53). To test whether the SSB plasmid triggers ATR activation in NPE, it was found that no detectable Chk1 phosphorylation was induced by SSB plasmid in NPE (FIG. 9B). It was reasoned that some phosphatases of Chk1 in NPE may destabilize the potential Chk1 phosphorylation by activated ATR. To test this possibility, tautomycin was used, which has been shown to stabilize Chk1 phosphorylation induced by AT70 in the LSS system (34). Interestingly, with the presence of tautomycin, the SSB plasmid triggered a very robust Chk1 phosphorylation, whereas CTL plasmid induced a minimal Chk1 phosphorylation in NPE (FIG. 9B). Both Chk1 phosphorylation events in NPE were inhibited by the addition of VE-822 (FIG. 9B). Whereas the minimal Chk1 phosphorylation induced by CTL plasmid in NPE may be due to increased DNA-to-cytoplasmic ratio (55), the SSB-induced increase of Chk1 phosphorylation suggests that ATR-Chk1 DDR is induced by SSB in NPE.

One striking feature of this experimental system is that SSB signaling is replication-independent in the HSS (FIGS. 8E and 8F), This is consistent with the deficiency of DNA replication elongation in the HSS system (37). Because a variety of checkpoint proteins play important roles for DNA replication, the defined SSB signaling system in a cell-free system of the present disclosure provides a powerful experimental system for future applications in determining direct roles of candidate checkpoint proteins in DDR pathway but not indirectly through their function in DNA replication. This replication-independent SSB-induced ATR-Chk1 DDR in the HSS system is reminiscent of the DSB-mimicking AT-70 induced replication-independent ATR-Chk1 DDR in the LSS system (34,49). It is believed that the ssDNA gap after SSB end resection is ˜18 nt-26 nt (FIG. 14B) and that the ssDNA gap is likely to be filled by DNA polymerase for SSB repair. Future work is needed to test whether such repair DNA synthesis is important for ATR activation.

The two modes of APE2-PCNA interaction are intriguing. APE2 interacts with PCNA's IDOL motif via its PIP box and associates with ssDNA via its Zf-GRF motif (24,30-32). Importantly, the present results demonstrated that APE2 Zf-GRF also interacts with PCNA, mainly through PCNA's CTM region. Therefore, two modes of APE2-PCNA interaction are proposed: APE2 PIP box-PCNA IDOL interaction and APE2 Zf-GRF-PCNA CTM interaction are designed as Mode I and Mode II interaction, respectively (FIG. 12A). Although it is not explicitly determined as of now how the two modes of APE2-PCNA interaction are selected and/or transitioned dynamically, a previous study in yeast showed that APE2 interacts with PCNA IDOL with the absence of DNA and switches to PCNA C-terminal tail upon entering a 3′ primer-template junction (31). The present findings identified that APE2 Zf-GRF interacts with PCNA CTM even in the absence of DNA (FIGS. 12A-E and 13A-E).

Furthermore, from these data, it appears that the two modes of APE2-PCNA interaction are neither required for nor mutually exclusive to each other. The present observations indicate that the Mode I interaction plays a major role in APE2 recruitment to SSB sites whereas the Mode II interaction plays an important role in APE2 activation in the HSS system. Nonetheless, the biological significance of Mode II of APE2-PCNA interaction is evidenced by deficiency of SSB end resection and SSB signaling by the mutant C470A APE2 in the HSS system (FIG. 14A). Why is the Mode II of APE2-PCNA interaction needed for APE2 activation in SSB signaling? One speculation is that the Mode II of APE2-PCNA interaction is needed to overcome the inhibition of APE2 by a previously unidentified negative regulator for SSB end resection in the HSS system. The Model I of APE2-PCNA interaction may bring APE2 to PCNA-bound DNA even under normal conditions; however, APE2 is not activated until the Mode II interaction makes appropriate confirmation change of the APE2-PCNA-DNA complex to stimulate APE2′s exonuclease activity. Notably, the ssDNA interaction via APE2's Zf-GRF is also important for APE2 activation (FIG. 4) (56). These three distinct mechanisms of APE2 recruitment and activation appear to ensure that SSB end resection only takes place at the right SSB sites for genome stability (FIG. 4).

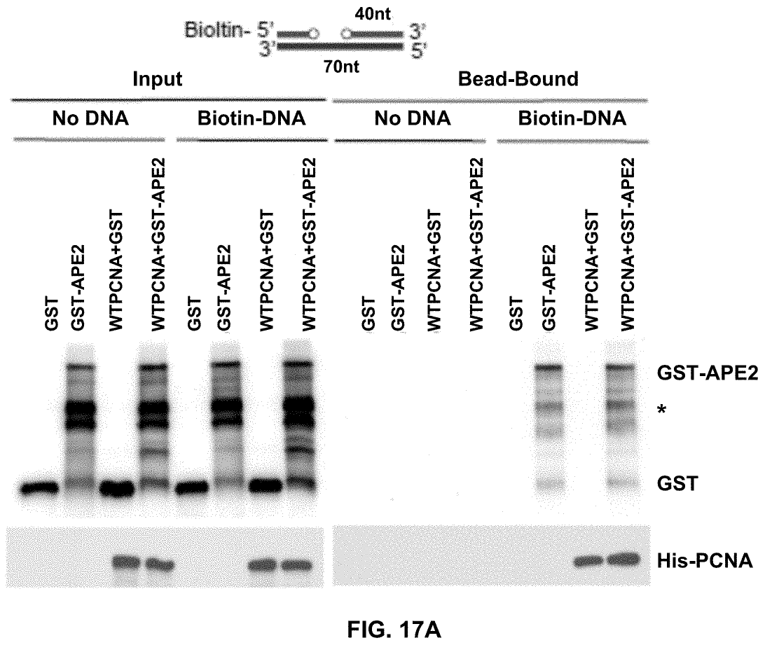

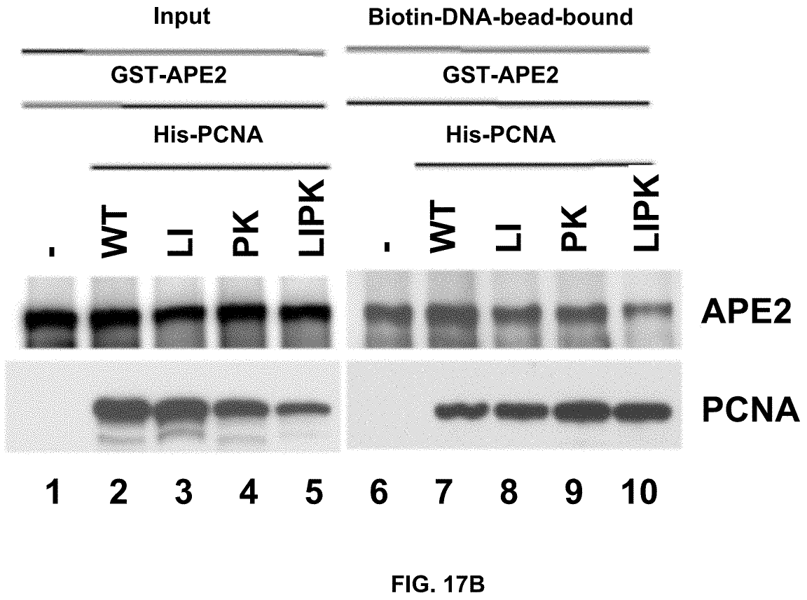

In addition, both IDOL and CTM regions within PCNA are important for PCNA-stimulated 3′-5′ exonuclease activity of APE2 in vitro (FIG. 16A). To examine the interaction of PCNA and APE2 to gapped DNA structure, an in vitro protein-DNA binding approach was established with biotin-gapped dsDNA coupled to streptavidin dynabeads (FIG. 17A). Notably, GST-APE2, but not GST, was found on Biotin-DNA-coupled beads but not “no DNA” beads, suggesting that APE2 binds to gapped DNA substrate in vitro at least under these experimental conditions. It is believed that the 1-3 nt ssDNA gap may be sufficient for APE2 interaction in vitro. Interestingly, the addition of WT PCNA has minimal effect on APE2's binding to the gapped DNA substrate (FIG. 17A). Furthermore, LI PCNA, PK PCNA, and LIPK PCNA behave similar to WT PCNA concerning APE2′s binding to the gapped DNA structure (FIG. 17B). It is speculated that both modes of PCNA-APE2 interaction are important for APE2 exonuclease activity in vitro. More structural studies can be implemented to figure out how exactly APE2, PCNA, and DNA interact with each other in a dynamic and coordinated fashion.

Previous studies have demonstrated that the homologue residues of E34A and D273A from yeast and human cells are exonuclease defective APE2 mutants (27,57). Previous studies have demonstrated that the E34A APE2 and D273A APE2 failed to rescue the hydrogen peroxide-induced Chk1 phosphorylation in APE2-depleted LSS system (24). To verify the Xenopus E34A APE2 and D273A APE2 are indeed exonuclease deficient mutants, their exonuclease activity was tested using FAM-labeled gapped DNA structure as template in vitro. Consistent with previous studies on APE2 in other species, the PCNA-promoted exonuclease activity of Xenopus APE2 was significantly decreased in E34A APE2 and D273A APE2 (FIG. 15B). Notably, WT, but not E34A or D273A, Myc-APE2 rescued Chk1 phosphorylation in APE2-depleted HSS (“extract” panel, FIG. 15C). Although E34A and D273A APE2 associated with DNA in a similar fashion as WT APE2, the recruitment of RPA32 and checkpoint protein complex including ATR, ATRIP, TopBP1, and Rad9 onto SSB plasmid was rescued by WT, but not E34A or D273A, APE2 in APE2-depleted HSS (“DNA-bound” panel, FIG. 15C). These observations suggest that the exonuclease activity of APE2 is indeed important for the SSB-induced ATR activation in the HSS system.

APE2 Zf-GRF interaction with PCNA is a distinct feature compared with ssDNA interaction. There are three types of mutants in APE2 Zf-GRF in terms of PCNA and ssDNA interaction: (I) C470A Zf-GRF and F486A-Y487A Zf-GRF are deficient for PCNA interaction but proficient for ssDNA interaction, (II) R502A APE2 Zf-GRF is deficient for ssDNA interaction but proficient for PCNA interaction (32), and (III) G483A-R484A APE2 Zf-GRF is defective for both PCNA association and ssDNA interaction (FIGS. 12A-12E and FIGS. 13B-13E). These observations suggest that the PCNA association and ssDNA interaction of APE2 Zf-GRF are neither dependent on nor mutually exclusive to each other. The C470 residue of Zf-GRF is localized in the flexible region between Polyproline helix and β1 region (FIG. 12A), suggesting that the C470A point mutation may not affect the secondary structure of APE2 Zf-GRF. Because Zf-GRF has been found in more than 100 proteins involved in DNA/RNA metabolism such as NEIL3 and Top3 (32), the Mode II of APE2-PCNA interaction may be applicable to future structure-function studies of other Zf-GRF-containing proteins.

SSB end resection has unique characteristics in comparison to other DNA end processing pathways such as DSB end resection. In this study, substantial data is presented to show that a site-specific SSB structure triggers an ATR-Chk1 DDR pathway via SSB end resection in a eukaryotic cell-free system. One distinct feature of SSB end resection is the critical role of APE2's 3′-5′ exonuclease activity (FIG. 14D). Although a nicked dsDNA structure is resected by recombinant APE2 in vitro (27), the FAM-SSB structure is still catalyzed into Type II resected products by other DNA end processing enzymes in APE2-depleted HSS (FIG. 14D). There are two possible explanations: APE2 plays an important role in both the initiation and continuation of 3′-5′ SSB end resection in the HSS system, and another unknown 3′-5′ exonuclease may compensate the initiation of 3′-5′ SSB end resection in APE2-depleted HSS. Alternatively, a previously unidentified 3′-5′ exonuclease is needed to initiate SSB end resection followed by continuation of 3′-5′ SSB end resection by APE2. Of note, no apparent Type II resected products were observed in the time-course experiment (FIG. 14B). It is believed that the initiation phase of 3′-5′ SSB end resection takes time to complete, and that end resection continuation is much quicker as long as a short ssDNA gap is generated. More experiments can be implemented to test these different possibilities. It has been investigated extensively that DSB end resection in the 5′-3′ direction couples DSB repair and DDR pathways (18). Exo1-meidated 5′-3′ DSB end resection has been implicated in DSB repair, nucleotide excision repair (NER) and mismatch repair (MMR) pathways (58-60). Whereas Mre11 participates 3′-5′ end resection of protein-DNA adducts, a critical question that remains unanswered is whether the Mre11's 3′-5′ end resection plays a critical role for DDR pathway activation (61).

In addition, previous studies demonstrate that ATR and ATRIP preferentially bind to approximately 70 nt to 80 nt ssDNA coated with RPA in in vitro binding assays (10). Consistent with this, a gapped plasmid with 68 nt ssDNA is sufficient to trigger an ATR-Chk1 DDR pathway in a DNA-PKcs-dependent manner in human cell-free extracts (62). To test whether ATR and ATRIP are recruited to this short stretch of ssDNA (18 nt-26 nt), it was discovered that as short as 20 nt ssDNA was sufficient for binding of RPA, ATR and ATRIP in the HSS system (FIG. 14C). This observation is consistent with the preferential recruitment of ATR and ATRP to SSB sites in the HSS system shown in FIG. 14A. Future studies will focus on how the SSB end resection is terminated to promote SSB repair.

From COSMIC analysis (cancer.sanger.ac.uk/cancergenome/projects/cosmic) of 45 cancer patients with somatic mutations in APE2, 33 missense point mutations were found in APE2, out of which 21 mutant residues of human APE2 are identical to Xenopus APE2 in homologue analysis. These 21 substitution missense point mutants in human APE2 are converted into Xenopus APE2: G10E, T38S, V491, G51S, R62H, A69S, A79S, E83G, L110R, E152K, R159C, R208C, R244C, R264H, H300Q, A314T, E343K, A366V, G456E, E468G, and R484H. It was found that 15 residues are in the N-terminal EEP domain, and two mutant residues (E468 and R484) are in the Zf-GRF domain. In particular, the R484H mutant within APE2 Zf-GRF may be deficient for PCNA interaction and ssDNA interaction. In addition, one nonsense substitution in human APE2 (c.1342G>T, p.E448*) was confirmed as a somatic mutation in a lung carcinoma patient (TCGA-75-6211-01), leading to a mutant APE2 protein that lacks the whole Zf-GRF domain. These somatic mutations in cancer patients suggest that the Zf-GRF domain of APE2 may have biological significance via its checkpoint function. Dysfunctions in DNA repair and DDR signaling pathways are implicated in cancer development (6). Importantly, a variety of DNA repair and DDR proteins including ATR and Chk1 become therapeutic targets and are currently being tested in the laboratory and clinical studies (63). A better understanding of DDR pathway activation in response to SSBs has implications in new avenues of cancer treatment. Findings from these experiments will impact future translational studies such as anti-cancer therapies via the modulation of novel role of APE2 in SSB signaling using mammalian cell lines or mice models. Overall, the present evidence using defined SSB end resection and SSB signaling in Xenopus provides novel insights into SSB-induced DDR pathway by APE2 in a eukaryotic cell-free system.

Table 1 below provides information including key reagents and resources used in Example 1. Antibodies, chemicals, and recombinant DNA and proteins, critical commercial assays, and software are summarized in this table. Oligonucleotides used in this example are presented with the sequence information, below.

TABLE 1
key reagents and resources.

| | | |
|---|---|---|
| REAGENT or RESOURCE | SOURCE | IDENTIFIER |

Antibodies

| | | |
|---|---|---|
| Anti-APE2 | Willis et al., 2013 | N/A |
| Anti-ATR | Willis et al., 2013 | N/A |
| Anti-ATRIP | Willis et al., 2013 | N/A |
| Anti-Chk1 P-S345 | Cell Signaling Technology | Cat# 2348 |
| Anti-Chk1 | Santa Cruz | Cat# sc-7898 |
| Anti-Claspin | Lupardus et al., 2006 | N/A |
| Anti-GST | Santa Cruz | Cat# sc-138 |
| Anti-Histone 3 | Abcam | Cat# ab1791 |
| Anti-Myc | Santa Cruz | Cat# sc-40 |
| Anti-PARP1 | Ryu et al., 2010 | N/A |
| Anti-PCNA | Santa Cruz | Cat# sc-56 |
| Anti-RPA32 | Yan et al., 2009 | N/A |
| Anti-RPA32 P-S33 | Bethyl Laboratories | Cat# A300-246A |
| Anti-Rad9 | Jones et al., 2003 | N/A |
| Anti-TopBP1 | Yan et al., 2009 | N/A |
| Anti-XRCC1 | Lin et al., 2017 | N/A |
| | (Manuscript in preparation) | |
| Peroxidase-conjugated monoclonal mouse | Jackson ImmunoResearch | Cat# 211-032-171 |
| anti-rabbit IgG, light chain specific | Laboratories | |
| Goat anti-rabbit IgG (H + L)-HRP | Thermo Fisher Scientific | Cat# 31460 |
| Goat anti-mouse IgG-HRP | Santa Cruz | Cat# sc-2031 |

Chemicals, Peptides, and Recombinant Proteins

| | | |
|---|---|---|
| 4-Amino-1,8-naphthalimide (4-AN) | Sigma | Cat# A0966 |
| Calf intestine phosphatase (CIP) | New England BioLabs | Cat# M0290 |
| Dynabeads M-280 Streptavidin | Invitrogen | Cat# 112.06D |
| Geminin | Yan et al., 2009 | N/A |
| Glutathione Sepharose Fast Flow | GE Healthcare | Cat# 17-5132-01 |
| Human chorionic gonadotropin (HCG) | Sigma | Cat# CG10 |
| KU-55933 | CalBiochem | Cat# 118500 |
| Nit-NTA Agarose | Qiagen | Cat# 1018244 |
| NU7441 | Selleckchem | Cat# S2638 |
| Nt. BstNBI | New England BioLabs | Cat# R0607 |
| PageRuler prestained protein ladder (10-180kD) | Thermo Fisher Scientific | Cat# 26616 |
| Phenol-Chloroform | CalBiochem | Cat# 6810 |
| Pregnant Mare Serum Gonadotropin (PMSG) | BMD Millipore | Cat# 36-722-25000I |
| rProtein A Sepharose Fast Flow | GE Healthcare | Cat# 17-1279-01 |
| Recombinant GST protein | Homemade | N/A |
| Sbfl-HF | New England BioLabs | Cat# R3642 |
| VE-822 | Selleckchem | Cat# S7102 |
| WesternBright ECL | Advansta | Cat# K-12045 |
| WesternBright Sirius | Advansta | Cat# K-12043 |
| [³²P-α]-dATP | PerkinElmer | Cat# BLU512Z500UC |

Critical Commercial Assays

| | | |
|---|---|---|
| QuikChange II XL site-directed mutagenesis kit | Agilent Technologies | Cat# 200521 |
| KOD Hot Start DNA polymerase PCR kit | EMD Millipore | Cat# 71086 |
| TNT SP6 Quick Coupled | Promega | Cat# L2080 |
| Transcription/Translation System | | |
| MinElute reaction cleanup kit | Qiagen | Cat# 28206 |
| Qiagen plasmid midi kit | Qiagen | Cat# 12143 |
| QIAprep spin miniprep kit | Qiagen | Cat# 27106 |
| QIAquick gel extraction kit | Qiagen | Cat# 28706 |

Recombinant DNA

| | | |
|---|---|---|
| pUC19 | New England Biolabs | Cat# N3041S |
| xIPCNA | Thermo Fisher | Cat# MXL1736-202772935 |
| xIXRCC1 | Source BioScience | Cat# IRBHp990H078D |
| pGEX-4T1-APE2 | Willis et al., 2013 | N/A |
| pGEX-4T1-WT APE2-ZF | Lin et al., 2017 | N/A |
| | (Manuscript in preparation) | |
| pGEX-4T1-G483A-R484A APE2-ZF | Lin et al., 2017 | N/A |
| | (Manuscript in preparation) | |
| pGEX-4T1-F486A-Y487A APE2-ZF | Lin et al., 2017 | N/A |
| | (Manuscript in preparation) | |
| pGEX-4T1-C470A APE2-ZF | Lin et al., 2017 | N/A |
| | (Manuscript in preparation) | |
| pGEX-4T1-R502A APE2-ZF | Lin et al., 2017 | N/A |
| | (Manuscript in preparation) | |
| pGEX-4T1-XRCC1 | Lin et al., 2017 | N/A |
| | (Manuscript in preparation) | |
| pET28a-WT PCNA | Lin et al., 2017 | N/A |
| | (Manuscript in preparation) | |
| pET28a-LI PCNA | Lin et al., 2017 | N/A |
| | (Manuscript in preparation) | |
| pET28a-PK PCNA | Lin et al., 2017 | N/A |
| | (Manuscript in preparation) | |
| pET28a-LIPK PCNA | Lin et al., 2017 | N/A |
| | (Manuscript in preparation) | |
| pCS2-MT-WT APE2 | Willis et al., 2013 | N/A |
| pCS2-MT-C470A APE2 | Lin et al., 2017 | N/A |
| | (Manuscript in preparation) | |
| pCS2-MT-G483A-R484A APE2 | Lin et al., 2017 | N/A |
| | (Manuscript in preparation) | |

Software and Algorithms

| | | |
|---|---|---|
| ImageQuant software | GE Healthcare | N/A |

### Example 2

### A small molecule Celastrol Compromises the Binding of APE2 Zf-GRF to ssDNA, APE2 Exonuclease Activity, and Defined SSB-Induced DDR Pathway in the HSS System

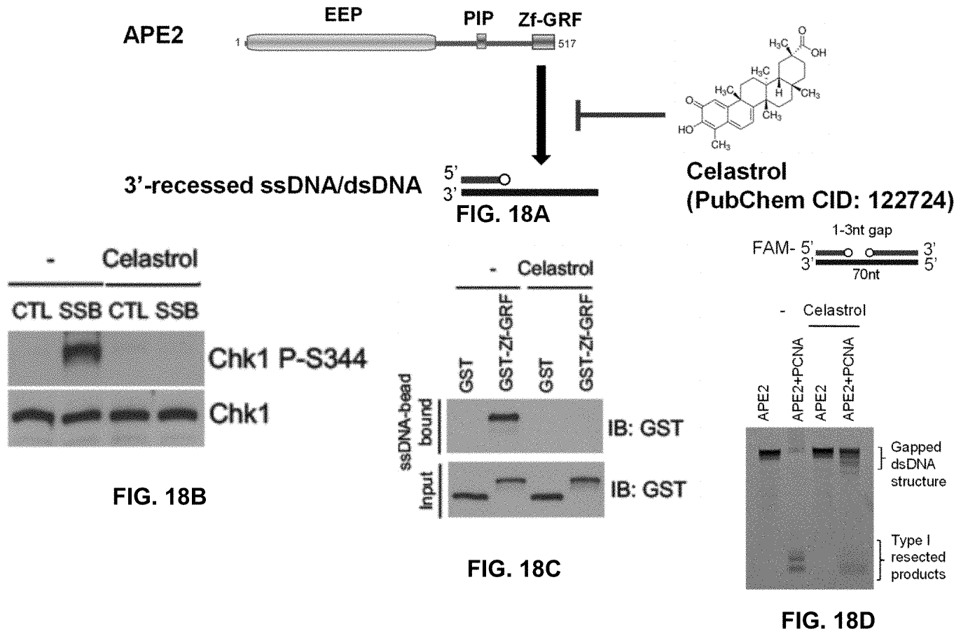

As demonstrated in Example 1 above, APE2 is composed of three conserved domains: A N-terminal endonuclease/exonuclease/phosphatase (EEP) domain, a middle PIP box domain, and a C-terminal Zf-GRF domain (FIG. 18A). Our recent studies and Example 1, above, show that APE2 Zf-GRF preferentially associates with ssDNA and that the Zf-GRF-ssDNA binding is critical for APE2's exonuclease activity and functions in SSB signaling following oxidative stress (Wallace et al., 2017).

Celastrol is a quinone methide triterpene from Tripterygium wilfordii (also known as Thunder of God Vine) that has been used as a natural medicine in China for many years (Yang et al., 2006). Accumulating evidence suggests that Celastrol exhibits anti-tumor activities in a variety of different types of cancers, including prostate cancer (Dai et al., 2009; Yang et al., 2006), breast cancer (Raja et al., 2014; Shrivastava et al., 2015), pancreatic cancer (Yadav et al., 2010), lung cancer (Liu et al., 2014; Wang et al., 2015), and glioblastoma (Boridy et al., 2014). Numerous molecule targets of Celastrol have been identified, such as IKK-alpha, IKK-beta, cdc37, p23, heat short factor-1, and proteamsomes (Chadli et al., 2010; Lee et al., 2006; Sreeramulu et al., 2009; Wang et al., 2015). However, it remains unknown whether Celastrol plays a role in the SSB signaling and repair pathways.

Example 1 demonstrated that GST-APE2 ZF can bind to ssDNA in vitro (FIG. 12E). The present example is the first demonstration that Celastrol inhibited the binding of APE2 Zf-GRF to ssDNA in vitro (FIG. 18C). It was shown that the SSB plasmid induces Chk1 phosphorylation in the HSS system in Example 1 (FIGS. 8 and 10). The addition of Celastrol to HSS system impaired the defined SSB-induced Chk1 phosphorylation (FIG. 18B). These observations suggest that Celastrol has a distinct role in preventing the binding of APE2 Zf-GRF to ssDNA and APE2's critical function in SSB signaling in the HSS system. In addition, APE2 promotes the PCNA-mediated end resection of a FAM-labeled gapped DNA structure via its 3′-5′ exonuclease activity in vitro (FIG. 16B in Example 1). Notably, Celastrol compromises the PCNA-mediated end resection of 3′-recessed DNA structure in vitro (FIG. 18D). All these findings strongly suggest that Celastrol binds to Zf-GRF within APE2 to prevent its binding to ssDNA and its 3′-5′ exonuclease activity, thereby leading to defective SSB signaling and genome instability. From these results, it can be concluded that Celastrol acts as an APE2 small molecule inhibitor to suppress its exonuclease activity and thus suppress SSB DDR activity.

### Example 3

### Identification of Small Molecule Compound Inhibitors of Single-Strand Break Signaling Via a Forward Chemical Genetic Screen

The above-described SSB signaling technology has established a tractable experimental system to investigate all aspects of the SSB signaling. One particular significant application of the SSB technology is to identify small molecule inhibitors of APE2 functions in the SSB signaling via a forward chemical genetic screen, followed by validation via established functional analyses described above. The present example describes systems using the SSB/HSS systems of the present disclosure for screening compound libraries for modulators (e.g., enhancers or inhibitors) of SSB DDR activity.

Screening Small Molecule Libraries and Identify New Inhibitor of APE2 in SSB Signaling.

A forward chemical genetic screen approach is used to identify small molecule inhibitors of APE2 functions in SSB signaling from available small molecule libraries as illustrated in FIG. 5 (discussed briefly in the description above). Example libraries include, but are not limited to, the DIVERSet Library (ChemBridge Corporation, San Diego, Calif.), which offers a diverse set of up to 50,000 small molecule compounds with extensive pharmacophore coverage. This small molecule library has been used extensively by a number of small molecule screenings, such as screen of Mre11 inhibitor Mirin and p53 inhibitor pifithrin-alpha (Dupre et al., 2008; Komarov et al., 1999).

Briefly, SSB plasmid is added to Xenopus HSS and transferred to 96-well plates containing 88 compounds (columns 1-11) per plate. A12, B12, and C12 are used for positive controls with phosphorylatable Chk1 peptide. D12, E12, and F12 are used for negative controls with non-phosphorylatable control peptide. ATR inhibitor VE-822 is used as a control in positions G12 and H12. The activity of ATR was examined by measuring the incorporation of radiolabeled [gamma-³²P]-ATP into a peptide derived from Chk1. The reactions are transferred to a 96-well p81 phosphocellulose plate, washed, dried, and exposed to a Phosphorlmager screen for visualization and quantification. The phosphorylation of Chk1 peptide is calculated for each sample according to the following formula: (value of sample—average of value of negative controls with control peptide)/(average value of 88 samples—average value of negative control). Percentages of inhibition of Chk1 peptide phosphorylation are calculated according to following formula: (1- (average of sample value—average value of negative controls)/(average value of positive controls—average value of negative controls))×100.

Validating the Identified Small Molecule Inhibitors of APE2 Functions in SSB Signaling.

Once hit small molecules are identified, the inhibitory effect of these molecules will be validated via several established approaches: (1) APE2's 3′-5′ exonuclease activity in vitro using the method; (2) The binding of APE2 Zf-GRF to ssDNA; (3) DNA end resection of FAM-dsDNA-SSB in the HSS system; (4) and the defined SSB-induced ATR-Chk1 DDR pathway activation in the HSS system, which are described in Example 1, above.

### Sequence Information

The following provides a brief description of oligonucleotide (DNA/RNA) and peptide sequences referred to in the present disclosure. This list may not be exhaustive and other sequences may be referred to by designations known to those of skill in the art.

DNA sequence (+ strand) of pUC19 plasmid
SEQ ID NO: 1
5′-TCGCGCGTTTCGGTGATGACGGTGAAAACCTCTGACACATGCAGCTCCCGGAGAC
GGTCACAGCTTGTCTGTAAGCGGATGCCGGGAGCAGACAAGCCCGTCAGGGCGC
GTCAGCGGGTGTTGGCGGGTGTCGGGGCTGGCTTAACTATGCGGCATCAGAGCA
GATTGTACTGAGAGTGCACCATATGCGGTGTGAAATACCGCACAGATGCGTAAGGA
GAAAATACCGCATCAGGCGCCATTCGCCATTCAGGCTGCGCAACTGTTGGGAAGG
GCGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAAAC
GACGGCCAGTGAATTCGAGCTCGGTACCCGGGGATCCTCTAGAGTCGACCTGCAG
GCATGCAAGCTTGGCGTAATCATGGTCATAGCTGTTTCCTGTGTGAAATTGTTATCC
GCTCACAATTCCACACAACATACGAGCCGGAAGCATAAAGTGTAAAGCCTGGGGTG
CCTAATGAGTGAGCTAACTCACATTAATTGCGTTGCGCTCACTGCCCGCTTTCCAGT
CGGGAAACCTGTCGTGCCAGCTGCATTAATGAATCGGCCAACGCGCGGGGAGAGG
CGGTTTGCGTATTGGGCGCTCTTCCGCTTCCTCGCTCACTGACTCGCTGCGCTCG
GTCGTTCGGCTGCGGCGAGCGGTATCAGCTCACTCAAAGGCGGTAATACGGTTAT
CCACAGAATCAGGGGATAACGCAGGAAAGAACATGTGAGCAAAAGGCCAGCAAAA
GGCCAGGAACCGTAAAAAGGCCGCGTTGCTGGCGTTTTTCCATAGGCTCCGCCCC
CCTGACGAGCATCACAAAAATCGACGCTCAAGTCAGAGGTGGCGAAACCCGACAG
GACTATAAAGATACCAGGCGTTTCCCCCTGGAAGCTCCCTCGTGCGCTCTCCTGTT
CCGACCCTGCCGCTTACCGGATACCTGTCCGCCTTTCTCCCTTCGGGAAGCGTGG
CGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCG
GTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCT
TGAAGTGGTGGCCTAACTACGGCTACACTAGAAGAACAGTATTTGGTATCTGCGCT
CTGCTGAAGCCAGTTACCTTCGGAAAAAGAGTTGGTAGCTCTTGATCCGGCAAACA
AACCACCGCTGGTAGCGGTGGTTTTTTTGTTTGCAAGCAGCAGATTACGCGCAGAA
AAAAAGGATCTCAAGAAGATCCTTTGATCTTTTCTACGGGGTCTGACGCTCAGTGG
AACGAAAACTCACGTTAAGGGATTTTGGTCATGAGATTATCAAAAAGGATCTTCACC
TAGATCCTTTTAAATTAAAAATGAAGTTTTAAATCAATCTAAAGTATATATGAGTAAAC
TTGGTCTGACAGTTACCAATGCTTAATCAGTGAGGCACCTATCTCAGCGATCTGTCT
ATTTCGTTCATCCATAGTTGCCTGACTCCCCGTCGTGTAGATAACTACGATACGGGA
GGGCTTACCATCTGGCCCCAGTGCTGCAATGATACCGCGAGACCCACGCTCACCG
GCTCCAGATTTATCAGCAATAAACCAGCCAGCCGGAAGGGCCGAGCGCAGAAGTG
GTCCTGCAACTTTATCCGCCTCCATCCAGTCTATTAATTGTTGCCGGGAAGCTAGA
GTAAGTAGTTCGCCAGTTAATAGTTTGCGCAACGTTGTTGCCATTGCTACAGGCATC
GTGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGGTC
CTCCGATCGTTGTCAGAAGTAAGTTGGCCGCAGTGTTATCACTCATGGTTATGGCA
GCACTGCATAATTCTCTTACTGTCATGCCATCCGTAAGATGCTTTTCTGTGACTGGT
GAGTACTCAACCAAGTCATTCTGAGAATAGTGTATGCGGCGACCGAGTTGCTCTTG
CCCGGCGTCAATACGGGATAATACCGCGCCACATAGCAGAACTTTAAAAGTGCTCA
TCATTGGAAAACGTTCTTCGGGGCGAAAACTCTCAAGGATCTTACCGCTGTTGAGA
TCCAGTTCGATGTAACCCACTCGTGCACCCAACTGATCTTCAGCATCTTTTACTTTC
ACCAGCGTTTCTGGGTGAGCAAAAACAGGAAGGCAAAATGCCGCAAAAAAGGGAA
TAAGGGCGACACGGAAATGTTGAATACTCATACTCTTCCTTTTTCAATATTATTGAAG
CATTTATCAGGGTTATTGTCTCATGAGCGGATACATATTTGAATGTATTTAGAAAAAT
AAACAAATAGGGGTTCCGCGCACATTTCCCCGAAAAGTGCCACCTGACGTCTAAGA
AACCATTATTATCATGACATTAACCTATAAAAATAGGCGTATCACGAGGCCCTTTCG
TC -3′
DNA sequence (+ strand) of engineered pS plasmid
SEQ ID NO: 2
5′-TCGCGCGTTTCGGTGATGACGGTGAAAACCTCTGACACATGCAGCTCCCGGAGAC
GGTCACAGCTTGTCTGTAAGCGGATGCCGGGAGCAGACAAGCCCGTCAGGGCGC
GTCAGCGGGTGTTGGCGGGTGTCGGGGCTGGCTTAACTATGCGGCATCAGAGCA
GATTGTACTGAGAGTGCACCATATGCGGTGTGAAATACCGCACAGATGCGTAAGGA
GAAAATACCGCATCAGGCGCCATTCGCCATTCAGGCTGCGCAACTGTTGGGAAGG
GCGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAAAC
GACGGCCAGTGAATTCGAGCTCGGTACCCGGGGATCCTCTAGAGTCGACCTGCAG
GCATGCAAGCTTGGCGTAATCATGGTCATAGCTGTTTCCTGTGTGAAATTGTTATCC
GCTCACAATTCCACACAACATACGAGCCGGAAGCATAAAGTGTAAAGCCTGGGGTG
CCTAATGAGTGAGCTAACTCACATTAATTGCGTTGCGCTCACTGCCCGCTTTCCAGT
CGGGAAACCTGTCGTGCCAGCTGCATTAATGAATCGGCCAACGCGCGGGGAGAGG
CGGTTTGCGTATTGGGCGCTCTTCCGCTTCCTCGCTCACTGGCTCGCTGCGCTCG
GTCGTTCGGCTGCGGCGAGCGGTATCAGCTCACTCAAAGGCGGTAATACGGTTAT
CCACAGAATCAGGGGATAACGCAGGAAAGAACATGTGAGCAAAAGGCCAGCAAAA
GGCCAGGAACCGTAAAAAGGCCGCGTTGCTGGCGTTTTTCCATAGGCTCCGCCCC
CCTGACGAGCATCACAAAAATCGACGCTCAAGTCAGAGGTGGCGAAACCCGACAG
GACTATAAAGATACCAGGCGTTTCCCCCTGGAAGCTCCCTCGTGCGCTCTCCTGTT
CCGACCCTGCCGCTTACCGGATACCTGTCCGCCTTTCTCCCTTCGGGAAGCGTGG
CGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCG
GTAACTATCGTCTTGAATCCAACCCGGTAAGACACGACTTATCGCCACTGGCAGCA
GCCACTGGTAACAGGATTAGCAGAGCGAGGTATGTAGGCGGTGCTACAGAGTTCT
TGAAGTGGTGGCCTAACTACGGCTACACTAGAAGAACAGTATTTGGTATCTGCGCT
CTGCTGAAGCCAGTTACCTTCGGAAAAAGAGTTGGTAGCTCTTGATCCGGCAAACA
AACCACCGCTGGTAGCGGTGGTTTTTTTGTTTGCAAGCAGCAGATTACGCGCAGAA
AAAAAGGATCTCAAGAAGATCCTTTGATCTTTTCTACGGGGTCTGACGCTCAGTGG
AACGAAAACTCACGTTAAGGGATTTTGGTCATGAGATTATCAAAAAGGATCTTCACC
TAGATCCTTTTAAATTAAAAATGAAGTTTTAAATCAATCTAAAGTATATATGAGTAAAC
TTGGTCTGACAGTTACCAATGCTTAATCAGTGAGGCACCTATCTCAGCGATCTGTCT
ATTTCGTTCATCCATAGTTGCCTGGCTCCCCGTCGTGTAGATAACTACGATACGGG
AGGGCTTACCATCTGGCCCCAGTGCTGCAATGATACCGCGAGACCCACGCTCACC
GGCTCCAGATTTATCAGCAATAAACCAGCCAGCCGGAAGGGCCGAGCGCAGAAGT
GGTCCTGCAACTTTATCCGCCTCCATCCAGTCTATTAATTGTTGCCGGGAAGCTAG
AGTAAGTAGTTCGCCAGTTAATAGTTTGCGCAACGTTGTTGCCATTGCTACAGGCAT
CGTGGTGTCACGCTCGTCGTTTGGTATGGCTTCATTCAGCTCCGGTTCCCAACGAT
CAAGGCGAGTTACATGATCCCCCATGTTGTGCAAAAAAGCGGTTAGCTCCTTCGGT
CCTCCGATCGTTGTCAGAAGTAAGTTGGCCGCAGTGTTATCACTCATGGTTATGGC
AGCACTGCATAATTCTCTTACTGTCATGCCATCCGTAAGATGCTTTTCTGTGACTGG
TGAGTACTCAACCAAGTCATTCTGAGAATAGTGTATGCGGCGACCGAGTTGCTCTT
GCCCGGCGTCAATACGGGATAATACCGCGCCACATAGCAGAACTTTAAAAGTGCTC
ATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTT
CACCAGCGTTTCTGGGTGAGCAAAAACAGGAAGGCAAAATGCCGCAAAAAAGGGA
ATAAGGGCGACACGGAAATGTTGAATACTCATACTCTTCCTTTTTCAATATTATTGAA
GCATTTATCAGGGTTATTGTCTCATGAGCGGATACATATTTGAATGTATTTAGAAAAA
TAAACAAATAGGGGTTCCGCGCACATTTCCCCGAAAAGTGCCACCTGACGTCTAAG
AAACCATTATTATCATGACATTAACCTATAAAAATAGGCGTATCACGAGGCCCTTTC
GTC -3′
DNA sequence (+ strand) of nt 420 to 450 of pS plasmid (SEQ
ID NO: 2) (underlined residue
SEQ ID NO: 3
TCCTCTAGAGTCGACCTGCAGGCATGCAAGC
chemically synthesized peptide sequence of phosphorylatable
Chk1-derived peptide (bold residue is phosphorylatable
serine reside)
SEQ ID NO: 4
LVQGKGISFSQPACPDNML
chemically synthesized peptide sequence of non-phosphoryl-
atable Chk1-derived peptide (bold residue is non-phosphoryl-
atable serine reside)
SEQ ID NO: 5
LVQGKGISFAQPACPDNML
amino acid residues 456 to 517 of APE2 protein from Xenopus
laevis
SEQ ID NO: 6
GPPPPPNCKGHSEPCVLRTVKKAGPNCGRQFYVCCARPEGHSSNPQARCNFFLWLT
KKAGCED
amino acid residues 121-133 of PCNA protein (IDCL region)
from Xenopus
SEQ ID NO: 7
LDVEQLGIPEQEY
amino acid residues 251-261 of PCNA protein (CTM region)
from Xenopus
SEQ ID NO: 8
LAPKIEDEEAS

TABLE 2
additional oligonucleotide sequences used in the Examples, above.

| Oligonucleotides | Description | SEQ ID NO: |
|---|---|---|
| F-MutantSite4: | Chemically synthesized | SEQ ID NO: 9 |
| 5′-CGTTCATCCATAGTTGCCTGGCTCCCC | forward primer for | |
| GTCGTGTAGATAAC -3′ | mutant site 4 to make | |
| | engineered pS plasmid | |
| R-MutantSite4: | Chemically synthesized | SEQ ID NO: 10 |
| 5′-GTTATCTACACGACGGGGAGCCAGGC | reverse primer for | |
| AACTATGGATGAACG -3′ | mutant site 4 to make | |
| | engineered pS plasmid | |
| F-MutantSite3: | Chemically synthesized | SEQ ID NO: 11 |
| 5′-CCGCTTCCTCGCTCACTGGCTCGCTG | forward primer for | |
| CGCTCGGTCGTTC -3′ | mutant site 3 to make | |
| | engineered pS plasmid | |
| R-MutantSite3: | Chemically synthesized | SEQ ID NO: 12 |
| 5′-GAACGACCGAGCGCAGCGAGCCAGT | reverse primer for | |
| GAGCGAGGAAGCGG-3′ | mutant site 3 to make | |
| | engineered pS plasmid | |
| F-MutantSite2: | Chemically synthesized | SEQ ID NO: 13 |
| 5′- GGTAACTATCGTCTTGAATCCAACCC | forward primer to make | |
| GGTAAGACACG -3′ | mutant site 2 to make | |
| | engineered pS plasmid | |
| R-MutantSite2: | Chemically synthesized | SEQ ID NO: 14 |
| 5′- CGTGTCTTACCGGGTTGGATTCAAGA | reverse primer to make | |
| CGATAGTTACC -3′ | mutant site 2 to make | |
| | engineered pS plasmid | |
| F-FAM-SSB: | Chemically synthesized | SEQ ID NO: 15 |
| 6-FAM-5′-TCGGTACCCGGGGATCCTCTA | fluorescently labeled | |
| G-3′ | forward primer to make | |
| | engineered 70 bp FAM- | |
| | SSB structure | |
| R-FAM-SSB: | Chemically synthesized | SEQ ID NO: 16 |
| 5′-ACAGCTATGACCATGATTACG-3′ | fluorescently labeled | |
| | reverse primer to make | |
| | engineered 70 bp FAM- | |
| | SSB structure | |
| F-PCNA: | Chemically synthesized | SEQ ID NO: 17 |
| 5′-GGGGGGGGATCCATGTTTGAGGCTC | forward primer for | |
| GCTTGGTGCAGG-3′ | PCNA protein | |
| R-PCNA: | Chemically synthesized | SEQ ID NO: 18 |
| 5′-GGGGGGCGGCCGCTTAAGAAGCTTC | reverse primer for | |
| TTCATCTTCAATCTTG-3′ | PCNA protein | |
| F-XRCC1: | Chemically synthesized | SEQ ID NO: 19 |
| 5′-GGGGGGGAATTCATGCCTGTGATCA | forward primer for | |
| AACTGAAG-3′ | XRCC1 protein | |
| R-XRCC1: | Chemically synthesized | SEQ ID NO: 20 |
| 5′-GGGGGGCTCGAGTTACGCCTTGGGC | reverse primer for | |
| ACCACAACG-3′ | XRCC1 protein | |
| 80 nt-ssDNA: | Chemically synthesized | SEQ ID NO: 21 |
| Biotin-5′-GGTCGACTCTAGAGGATCCCCG | biotin labeled 80 nt | |
| GGTACCGAGCTCGAATTCACTGGCCGTC | long section of single | |
| GTTTTACAACGTCGTGACTGGGAAAACCCT-3′ | strand DNA | |
| 60 nt-ssDNA: | Chemically synthesized | SEQ ID NO: 22 |
| Biotin-5′-GGTCGACTCTAGAGGATCCCCG | biotin labeled 60 nt | |
| GGTACCGAGCTCGAATTCACTGGCCGTC | long section of single | |
| GTTTTACAAC-3′ | strand DNA | |
| 40 nt-ssDNA: | Chemically synthesized | SEQ ID NO: 23 |
| Biotin-5′-GGTCGACTCTAGAGGATCCCCG | biotin labeled 40 nt | |
| GGTACCGAGCTCGAATTC-3′ | long section of single | |
| | strand DNA | |
| 20 nt-ssDNA: | Chemically synthesized | SEQ ID NO: 24 |
| Biotin-5′-GGTCGACTCTAGAGGATCCC-3′ | biotin labeled 20 nt | |
| | long section of single | |
| | strand DNA | |
| 10 nt-ssDNA: | Chemically synthesized | SEQ ID NO: 25 |
| Biotin-5′-GGTCGACTCT-3′ | biotin labeled 10 nt | |
| | long section of single | |
| | strand DNA | |

### REFERENCES (incorporated herein by reference only where indicated in the specification above)

1. Caldecott, K. W. (2008) Single-strand break repair and genetic disease. Nat Rev Genet, 9, 619-631.
2. Yan, S., Sorrell, M. and Berman, Z. (2014) Functional interplay between ATM/ATR-mediated DNA damage response and DNA repair pathways in oxidative stress. Cell Mol Life Sci, 71, 3951-3967.
3. Andres, S. N., Schellenberg, M. J., Wallace, B. D., Tumbale, P. and Williams, R. S. (2015) Recognition and repair of chemically heterogeneous structures at DNA ends. Environ Mol Mutagen, 56, 1-21.
4. Lee, D. H., Liu, Y., Lee, H. W., Xia, B., Brice, A. R., Park, S. H., Balduf, H., Dominy, B. N. and Cao, W. (2015) A structural determinant in the uracil DNA glycosylase superfamily for the removal of uracil from adenine/uracil base pairs. Nucleic Acids Res, 43, 1081-1089.
5. Davis, L. and Maizels, N. (2014) Homology-directed repair of DNA nicks via pathways distinct from canonical double-strand break repair. Proc Natl Acad Sci USA, 111, E924-932.
6. Jackson, S. P. and Bartek, J. (2009) The DNA-damage response in human biology and disease. Nature, 461, 1071-1078.
7. Ciccia, A. and Elledge, S. J. (2010) The DNA damage response: making it safe to play with knives. Mol Cell, 40, 179-204.
8. Cimprich, K. A. and Cortez, D. (2008) ATR: an essential regulator of genome integrity. Nat Rev Mol Cell Biol, 9, 616-627.
9. Smith, K. D., Fu, M. A. and Brown, E.J. (2009) Tim-Tipin dysfunction creates an indispensible reliance on the ATR-Chk1 pathway for continued DNA synthesis. J Cell Biol, 187, 15-23.
10. Zou, L. and Elledge, S. J. (2003) Sensing DNA damage through ATRIP recognition of RPA-ssDNA complexes. Science, 300, 1542-1548.
11. Yan, S. and Michael, W. M. (2009) TopBP1 and DNA polymerase-alpha directly recruit the 9-1-1 complex to stalled DNA replication forks. J Cell Biol, 184, 793-804.
12. Wang, J., Gong, Z. and Chen, J. (2011) MDC1 collaborates with TopBP1 in DNA replication checkpoint control. J Cell Biol, 193, 267-273.
13. Acevedo, J., Yan, S. and Michael, W. M. (2016) Direct Binding to Replication Protein A (RPA)-coated Single-stranded DNA Allows Recruitment of the ATR Activator TopBP1 to Sites of DNA Damage. J Biol Chem, 291, 13124-13131.
14. Marechal, A. and Zou, L. (2015) RPA-coated single-stranded DNA as a platform for post-translational modifications in the DNA damage response. Cell Res, 25, 9-23.
15. Matsuoka, S., Ballif, B. A., Smogorzewska, A., McDonald, E. R., 3rd, Hurov, K. E., Luo, J., Bakalarski, C. E., Zhao, Z., Solimini, N., Lerenthal, Y. et al. (2007) ATM and ATR substrate analysis reveals extensive protein networks responsive to DNA damage. Science, 316, 1160-1166.
16. Guo, Z., Kumagai, A., Wang, S. X. and Dunphy, W. G. (2000) Requirement for Atr in phosphorylation of Chk1 and cell cycle regulation in response to DNA replication blocks and UV-damaged DNA in Xenopus egg extracts. Genes Dev, 14, 2745-2756.
17. Bakkenist, C. J. and Kastan, M. B. (2003) DNA damage activates ATM through intermolecular autophosphorylation and dimer dissociation. Nature, 421, 499-506.
18. Symington, L. S. and Gautier, J. (2011) Double-strand break end resection and repair pathway choice. Annu Rev Genet, 45, 247-271.
19. You, Z. and Bailis, J. M. (2010) DNA damage and decisions: CtIP coordinates DNA repair and cell cycle checkpoints. Trends Cell Biol, 20, 402-409.
20. Guo, Z., Kozlov, S., Lavin, M. F., Person, M. D. and Paull, T. T. (2010) ATM activation by oxidative stress. Science, 330, 517-521.
21. Khoronenkova, S. V. and Dianov, G. L. (2015) ATM prevents DSB formation by coordinating SSB repair and cell cycle progression. Proc Natl Acad Sci USA, 112, 3997-4002.
22. Richardson, C. and Jasin, M. (2000) Frequent chromosomal translocations induced by DNA double-strand breaks. Nature, 405, 697-700.
23. Rudin, N. and Haber, J. E. (1988) Efficient repair of HO-induced chromosomal breaks in Saccharomyces cerevisiae by recombination between flanking homologous sequences. Mol Cell Biol, 8, 3918-3928.
24. Willis, J., Patel, Y., Lentz, B. L. and Yan, S. (2013) APE2 is required for ATR-Chk1 checkpoint activation in response to oxidative stress. Proc Natl Acad Sci USA, 110, 10592-10597.
25. Okano, S., Lan, L., Caldecott, K. W., Mori, T. and Yasui, A. (2003) Spatial and temporal cellular responses to single-strand breaks in human cells. Mol Cell Biol, 23, 3974-3981.
26. Tell, G., Quadrifoglio, F., Tiribelli, C. and Kelley, M. R. (2009) The many functions of APE1/Ref-1: not only a DNA repair enzyme. Antioxid Redox Sign, 11, 601-620.
27. Burkovics, P., Szukacsov, V., Unk, I. and Haracska, L. (2006) Human Ape2 protein has a 3′-5′ exonuclease activity that acts preferentially on mismatched base pairs. Nucleic Acids Res, 34, 2508-2515.
28. Guikema, J. E., Gerstein, R. M., Linehan, E. K., Cloherty, E. K., Evan-Browning, E., Tsuchimoto, D., Nakabeppu, Y. and Schrader, C. E. (2011) Apurinic/apyrimidinic endonuclease 2 is necessary for normal B cell development and recovery of lymphoid progenitors after chemotherapeutic challenge. J lmmunol, 186, 1943-1950.
29. Moldovan, G. L., Pfander, B. and Jentsch, S. (2007) PCNA, the maestro of the replication fork. Cell, 129, 665-679.
30. Burkovics, P., Hajdu, I., Szukacsov, V., Unk, I. and Haracska, L. (2009) Role of PCNA-dependent stimulation of 3′-phosphodiesterase and 3′-5′ exonuclease activities of human Ape2 in repair of oxidative DNA damage. Nucleic Acids Res, 37, 4247-4255.
31. Unk, I., Haracska, L., Gomes, X. V., Burgers, P. M. J., Prakash, L. and Prakash, S. (2002) Stimulation of 3′->5′ exonuclease and 3′phosphodiesterase activities of yeast Apn2 by proliferating cell nuclear antigen. Mol Cell Biol, 22, 6480-6486.
32. Wallace, B. D., Berman, Z., Mueller, G. A., Lin, Y., Chang, T., Andres, S. N., Wojtaszek, J. L., DeRose, E. F., Appel, C. D., London, R. E. et al. (2017) APE2 Zf-GRF facilitates 3′-5′ resection of DNA damage following oxidative stress. Proc Natl Acad Sci USA, 114, 304-309.
33. Lupardus, P. J., Byun, T., Yee, M. C., Hekmat-Nejad, M. and Cimprich, K. A. (2002) A requirement for replication in activation of the ATR-dependent DNA damage checkpoint. Genes Dev, 16, 2327-2332.
34. Kumagai, A. and Dunphy, W. G. (2000) Claspin, a novel protein required for the activation of Chk1 during a DNA replication checkpoint response in Xenopus egg extracts. Mol Cell, 6, 839-849.
35. Lebofsky, R., Takahashi, T. and Walter, J. C. (2009) DNA replication in nucleus-free Xenopus egg extracts. Methods Mol Biol, 521, 229-252.
36. Cupello, S., Richardson, C. and Yan, S. (2016) Cell-free Xenopus egg extracts for studying DNA damage response pathways. The International journal of developmental biology, 60, 229-236.
37. Walter, J., Sun, L. and Newport, J. (1998) Regulated chromosomal DNA replication in the absence of a nucleus. Mol Cell, 1, 519-529.
38. Philpott, A. and Yew, P. R. (2008) The Xenopus cell cycle: an overview. Mol Biotechnol, 39, 9-19.
39. Willis, J., Destephanis, D., Patel, Y., Gowda, V. and Yan, S. (2012) Study of the DNA damage checkpoint using Xenopus egg extracts. J Vis Exp, e4449.
40. Bai, L., Michael, W. M. and Yan, S. (2014) Importin beta-dependent nuclear import of TopBP1 in ATR-Chk1 checkpoint in Xenopus egg extracts. Cell Signal, 26, 857-867.
41. Lupardus, P. J. and Cimprich, K. A. (2006) Phosphorylation of Xenopus Rad1 and Hus1 defines a readout for ATR activation that is independent of Claspin and the Rad9 carboxy terminus. Mol Biol Cell, 17, 1559-1569.
42. Jones, R. E., Chapman, J. R., Puligilla, C., Murray, J. M., Car, A. M., Ford, C. C. and Lindsay, H. D. (2003) XRad17 is required for the activation of XChk1 but not XCds1 during checkpoint signaling in Xenopus. Mol Biol Cell, 14, 3898-3910.
43. Ryu, H., Al-Ani, G., Deckert, K., Kirkpatrick, D., Gygi, S. P., Dasso, M. and Azuma, Y. (2010) PIASy mediates SUMO-2/3 conjugation of poly(ADP-ribose) polymerase 1 (PARP1) on mitotic chromosomes. J Biol Chem, 285, 14415-14423.
44. Tsuchimoto, D., Sakai, Y., Sakumi, K., Nishioka, K., Sasaki, M., Fujiwara, T. and Nakabeppu, Y. (2001) Human APE2 protein is mostly localized in the nuclei and to some extent in the mitochondria, while nuclear APE2 is partly associated with proliferating cell nuclear antigen. Nucleic Acids Res, 29, 2349-2360.
45. Wilson, D. M., 3rd. (2003) Properties of and substrate determinants for the exonuclease activity of human apurinic endonuclease Apel. J Mol Biol, 330, 1027-1037.
46. Stokes, M. P., Van Hatten, R., Lindsay, H. D. and Michael, W. M. (2002) DNA replication is required for the checkpoint response to damaged DNA in Xenopus egg extracts. J Cell Biol, 158, 863-872.
47. Duursma, A. M., Driscoll, R., Elias, J. E. and Cimprich, K. A. (2013) A role for the MRN complex in ATR activation via TOPBP1 recruitment. Mol Cell, 50, 116-122.
48. Kumagai, A., Lee, J., Yoo, H. Y. and Dunphy, W. G. (2006) TopBP1 activates the ATR-ATRIP complex. Cell, 124, 943-955.
49. Yan, S., Lindsay, H. D. and Michael, W. M. (2006) Direct requirement for Xmus101 in ATR-mediated phosphorylation of Claspin bound Chk1 during checkpoint signaling. J Cell Biol, 173, 181-186.
50. Gamper, A. M., Rofougaran, R., Watkins, S. C., Greenberger, J. S., Beumer, J. H. and Bakkenist, C. J. (2013) ATR kinase activation in G1 phase facilitates the repair of ionizing radiation-induced DNA damage. Nucleic Acids Res, 41, 10334-10344.
51. Whitehouse, C. J., Taylor, R. M., Thistlethwaite, A., Zhang, H., Karimi-Busheri, F., Lasko, D. D., Weinfeld, M. and Caldecott, K. W. (2001) XRCC1 stimulates human polynucleotide kinase activity at damaged DNA termini and accelerates DNA single-strand break repair. Cell, 104, 107-117.
52. Eustermann, S., Wu, W. F., Langelier, M. F., Yang, J. C., Easton, L. E., Riccio, A. A., Pascal, J. M. and Neuhaus, D. (2015) Structural Basis of Detection and Signaling of DNA Single-Strand Breaks by Human PARP-1. Mol Cell, 60, 742-754.
53. MacDougall, C. A., Byun, T. S., Van, C., Yee, M. C. and Cimprich, K. A. (2007) The structural determinants of checkpoint activation. Genes Dev, 21, 898-903.
54. Ben-Yehoyada, M., Wang, L. C., Kozekov, I. D., Rizzo, C. J., Gottesman, M.E. and Gautier, J. (2009) Checkpoint signaling from a single DNA interstrand crosslink. Mol Cell, 35, 704-715.
55. Conn, C. W., Lewellyn, A. L. and Mailer, J. L. (2004) The DNA damage checkpoint in embryonic cell cycles is dependent on the DNA-to-cytoplasmic ratio. Dev Cell, 7, 275-281.
56. Lin, Y., Bai, L., Cupello, S., Deem, B., McLeod, M., Raj, J. and Yan, S. (2017) APE2 promotes DNA damage response from a single-strand break. Nucleic Acids Res. Under Revision.
57. Unk, I., Haracska, L., Prakash, S. and Prakash, L. (2001) 3′-phosphodiesterase and 3′->5′ exonuclease activities of yeast Apn2 protein and requirement of these activities for repair of oxidative DNA damage. Mol Cell Biol, 21, 1656-1661.
58. Lindsey-Boltz, L. A., Kemp, M. G., Reardon, J. T., Derocco, V., Iyer, R. R., Modrich, P. and Sancar, A. (2014) Coupling of human DNA excision repair and the DNA damage checkpoint in a defined in vitro system. J Biol Chem, 289, 5074-5082.
59. Garcia, V., Phelps, S. E., Gray, S. and Neale, M. J. (2011) Bidirectional resection of DNA double-strand breaks by Mre11 and Exo1. Nature, 479, 241-244.
60. Chen, X., Paudyal, S. C., Chin, R. I. and You, Z. (2013) PCNA promotes processive DNA end resection by Exo1. Nucleic Acids Res, 41, 9325-9338.
61. Deshpande, R. A., Lee, J. H., Arora, S. and Paull, T. T. (2016) Nbs1 Converts the Human Mre11/Rad50 Nuclease Complex into an Endo/Exonuclease Machine Specific for Protein-DNA Adducts. Mol Cell, 64, 593-606.
62. Vidal-Eychenie, S., Decaillet, C., Basbous, J. and Constantinou, A. (2013) DNA structure-specific priming of ATR activation by DNA-PKcs. J Cell Biol, 202, 421-429.
63. Curtin, N. J. (2012) DNA repair dysregulation from cancer driver to therapeutic target. Nat Rev Cancer, 12, 801-817.

1. A method for identifying modulators of single-strand break (SSB) DNA damage response (DDR) activity, the method comprising:
providing a composition comprising a plurality of engineered, site-specific, nicked, SSB plasmid structures, each having an inner (−) and outer (+) strand and genetically modified to comprise a single nick in the +strand only, the nick located at a single recognition site for a specific restriction enzyme and modified to have a hydroxyl group at each nicked end of the SSB plasmid structure;
providing a replication-independent, eukaryotic, cell-free extract capable of replication-independent initiation of one or more SSB DNA damage response (DDR) activities when incubated with the composition of engineered site-specific, nicked, SSB plasmid structures;
combining the composition of engineered, site-specific, nicked, SSB plasmid structures with the eukaryotic cell-free extract and a test compound to make a test mixture; and
detecting a SSB DDR activity.

2. The method of claim 1, wherein the replication-independent, eukaryotic, cell-free extract is a high-speed supernatant (HSS) from Xenopus egg extract.

3. The method of claim 2, wherein the HSS from Xenopus egg extract obtained by the following steps:
centrifuging Xenopus eggs at about 18,000-22,000 g for about 20-30min;
retaining a low-speed supernatant (LSS) layer;
centrifuging the LSS at about 240,000-280,000g, for about 90-120min; and
retaining the supernatant layer to produce the HSS.

4. The method of claim 1, wherein the one or more SSB DDR activities is selected from the group consisting of: initiation of DDR processes, recruitment of DDR signaling molecules, formation of DDR complexes, and repair of the engineered site-specific, SSB plasmid structure to form an intact circular plasmid.

5. The method of claim 1, wherein the one-or-more SSB DDR activities is selected from the group consisting of: APE2 activation, activation of an ATR complex, or both.

6. The method of claim 1, wherein detecting SSB DDR activity comprises detecting phosphorylation of a phosphorylatable peptide derived from a substrate of ATR kinase.

7. The method of claim 6, wherein the phosphorylatable peptide derived from a substrate of ATR kinase is a phosphorylatable Chk1-derived peptide.

8. The method of claim 7, wherein detecting phosphorylation of a phosphoylatable Chk1- derived peptide comprises detecting incorporation of radiolabeled ATP in to the Chk1-derived peptide.

9. The method of claim 7, wherein the phosphorylatable Chk1-derived peptide is a Chk-1 peptide having SEQ ID NO: 4.

10. The method of claim1, further comprising comparing the SSB DDR activity level in the presence of the test compound to the SSB DDR activity level in the absence of the test compound.

11. The method of claim 1, wherein the method is conducted on an array, the array comprising plurality of spots, wherein each spot receives the plurality of engineered site-specific, nicked, SSB plasmid structures, the replication-independent, eukaryotic, cell-free extract, and a detection substrate; wherein a portion of the plurality of spots independently receives a test compound.

12. The method of claim 11, wherein the replication-independent, eukaryotic, cell-free extract is a high-speed supernatant (HSS) from Xenopus egg extract.

13. The method of claim 11, wherein the detection substrate is a phosphorylatable peptide derived from a substrate of ATR kinase.

14. The method of claim 13, wherein the phosphorylatable peptide derived from a substrate of ATR kinase is a phosphorylatable Chk1-derived peptide.

15. The method of claim 14, wherein the detection substrate comprises a phosphorylatable Chk1-derived peptide having SEQ ID NO: 4.

16. The method of claim 11, wherein the detection substrate comprises a phosphorylatable Chk1-derived peptide and wherein phosphorylation of the phosphorylatable Chk1-derived peptide indicates occurrence of an SSB DDR activity in the test spot and wherein absence or reduced phosphorylation of the phosphorylatable Chk1-derived peptide in the test spot indicates that the test compound suppresses or inhibits an SSB DDR activity.

17. The method of claim 1, wherein an un-nicked plasmid corresponding to the engineered, site-specific, nicked, SSB plasmid structure comprises SEQ ID NO: 2.

18. The method of claim 1, wherein the plurality of engineered, site-specific, nicked, SSB plasmid structures each further comprises a single recognition site for a second restriction enzyme that is capable of creating a double strand break, wherein the nick of the SSB plasmid structure is located in recognition site for the second restriction enzyme, such that contact with the second restriction enzyme has no effect on the engineered, site-specific, nicked, SSB plasmid structure but produces a double strand break in an un-nicked plasmid corresponding to the engineered, site-specific, nicked, SSB plasmid structure.

19. The method of claim 18, wherein the un-nicked plasmid corresponding to the engineered, site-specific, nicked, SSB plasmid structure comprises SEQ ID NO: 3, SEQ ID NO: 3 having a single recognition site for each of restriction enzymes Nt.BstNBI and Sbfl, and wherein the un-nicked plasmid does not comprise any other recognition sites for restriction enzymes Nt.BstNBI or Sbfl.


SEQUENCE LISTING (25 sequences)

SEQ ID NO: 1 - DNA, 2686 bp, Artificial Sequence
  Sequence of + strand of pUC19 plasmid.
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 2686 bp, Artificial Sequence
  Sequence of + strand of engineered pS plasmid.
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactggctcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgaatccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctggctccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 3 - DNA, 31 bp, Artificial Sequence
  Sequence of + strand of nucleotides 420 to 450 of engineered pS plasmid having recognition site for restriction enzyme.
tcctctagagtcgacctgcaggcatgcaagc

SEQ ID NO: 4 - protein, 19 aa, Artificial Sequence
  Chemically synthesized peptide sequence of phosphorylatable Chk1-derived peptide.
LVQGKGISFSQPACPDNML

SEQ ID NO: 5 - protein, 19 aa, Artificial Sequence
  Chemically synthesized peptide sequence of non-phosphorylatable Chk1-derived peptide.
LVQGKGISFAQPACPDNML

SEQ ID NO: 6 - protein, 63 aa, Xenopus laevis
GPPPPPNCKGHSEPCVLRTVKKAGPNCGRQFYVCCARPEGHSSNPQARCNFFLWLTKKAGCED

SEQ ID NO: 7 - protein, 13 aa, Xenopus laevis
LDVEQLGIPEQEY

SEQ ID NO: 8 - protein, 11 aa, Xenopus laevis
LAPKIEDEEAS

SEQ ID NO: 9 - DNA, 41 bp, Artificial Sequence
  Chemically synthesized forward primer for mutant site 4 to make engineered pS plasmid.
cgttcatccatagttgcctggctccccgtcgtgtagataac

SEQ ID NO: 10 - DNA, 41 bp, Artificial Sequence
  Chemically synthesized reverse primer for mutant site 4 to make pS plasmid.
gttatctacacgacggggagccaggcaactatggatgaacg

SEQ ID NO: 11 - DNA, 39 bp, Artificial Sequence
  Chemically synthesized forward primer for mutant site 3 to make pS plasmid.
ccgcttcctcgctcactggctcgctgcgctcggtcgttc

SEQ ID NO: 12 - DNA, 39 bp, Artificial Sequence
  Chemically synthesized reverse primer for mutant site 3 to make engineered pS plasmid.
gaacgaccgagcgcagcgagccagtgagcgaggaagcgg

SEQ ID NO: 13 - DNA, 37 bp, Artificial Sequence
  Chemically synthesized forward primer to make mutant site 2 to make engineered pS plasmid.
ggtaactatcgtcttgaatccaacccggtaagacacg

SEQ ID NO: 14 - DNA, 37 bp, Artificial Sequence
  Chemically synthesized reverse primer to make mutant site 2 to make engineered pS plasmid.
cgtgtcttaccgggttggattcaagacgatagttacc

SEQ ID NO: 15 - DNA, 22 bp, Artificial Sequence
  Chemically synthesized fluorescently labeled forward primer to make engineered 70bp FAM-SSB structure.
tcggtacccggggatcctctag

SEQ ID NO: 16 - DNA, 21 bp, Artificial Sequence
  Chemically synthesized fluorescently labeled reverse primer to make engineered 70bp FAM-SSB structure.
acagctatgaccatgattacg

SEQ ID NO: 17 - DNA, 37 bp, Artificial Sequence
  Chemically synthesized forward primer for PCNA protein.
ggggggggatccatgtttgaggctcgcttggtgcagg

SEQ ID NO: 18 - DNA, 41 bp, Artificial Sequence
  Chemically synthesized reverse primer for PCNA protein.
ggggggcggccgcttaagaagcttcttcatcttcaatcttg

SEQ ID NO: 19 - DNA, 33 bp, Artificial Sequence
  Chemically synthesized forward primer for XRCC1 protein.
gggggggaattcatgcctgtgatcaaactgaag

SEQ ID NO: 20 - DNA, 34 bp, Artificial Sequence
  Chemically synthesized reverse primer for XRCC1 protein.
ggggggctcgagttacgccttgggcaccacaacg

SEQ ID NO: 21 - DNA, 80 bp, Artificial Sequence
  Chemically synthesized biotin labeled 80 nt long section of single strand DNA.
ggtcgactctagaggatccccgggtaccgagctcgaattcactggccgtcgttttacaacgtcgtgactgggaaaaccct

SEQ ID NO: 22 - DNA, 60 bp, Artificial Sequence
  Chemically synthesized biotin labeled 60 nt long section of single strand DNA.
ggtcgactctagaggatccccgggtaccgagctcgaattcactggccgtcgttttacaac

SEQ ID NO: 23 - DNA, 40 bp, Artificial Sequence
  Chemically synthesized biotin labeled 40 nt long section of single strand DNA.
ggtcgactctagaggatccccgggtaccgagctcgaattc

SEQ ID NO: 24 - DNA, 20 bp, Artificial Sequence
  Chemically synthesized biotin labeled 20 nt long section of single strand DNA.
ggtcgactctagaggatccc

SEQ ID NO: 25 - DNA, 10 bp, Artificial Sequence
  Chemically synthesized biotin labeled 10 nt long section of single strand DNA.
ggtcgactct